(19) United States
(12) Patent Application Publication
(10) Pub. No.: US 2024/0013384 A1
(43) Pub. Date: Jan. 11, 2024

(54) SYSTEMS, DEVICES, AND METHODS FOR PROVIDING DIAGNOSTIC ASSESSMENTS USING IMAGE ANALYSIS

(71) Applicant: Koios Medical, Inc., New York City, NY (US)

(72) Inventors: Lev BARINOV, Montclair, NJ (US); Ajit JAIRAJ, Edison, NJ (US)

(21) Appl. No.: 18/451,028

(22) Filed: Aug. 16, 2023

Related U.S. Application Data
(63) Continuation of application No. PCT/US2022/016865, filed on Feb. 17, 2022.
(60) Provisional application No. 63/232,976, filed on Aug. 13, 2021.
(60) Provisional application No. 63/150,286, filed on Feb. 17, 2021.

(51) Int. Cl. G06T 7/00; G16H 30/40

(52) U.S. Cl. CPC G06T 7/0012; G16H 30/40; G06T 2207/30096

(57) ABSTRACT

Embodiments disclosed include a method comprising receiving, at a first compute device, image data associated with a region of interest, a first diagnostic assessment associated with the image data, and a second diagnostic assessment associated with the image data, the second diagnostic assessment being different from the first diagnostic assessment. The method includes integrating the second diagnostic assessment with the first diagnostic assessment to generate a third diagnostic assessment associated with the clinical data.

### CROSS-REFERENCE TO RELATED APPLICATIONS

This application is a continuation of International Application No. PCT/US2022/016865, entitled “SYSTEMS, DEVICES, AND METHODS FOR PROVIDING DIAGNOSTIC ASSESSMENTS USING IMAGE ANALYSIS,” filed Feb. 17, 2022, which claims priority to U.S. Provisional Application No. 63/150,286 entitled “THE EFFICACY OF TI-RADS THROUGH ARTIFICIAL-INTELLIGENCE” filed Feb. 17, 2021, and to U.S. Provisional Application No. 63/232,976 entitled “SYSTEMS, DEVICES, AND METHODS FOR PROVIDING DIAGNOSTIC ASSESSMENTS USING IMAGE ANALYSIS” filed Aug. 13, 2021, the entire disclosures of which are incorporated herein by reference.

### FIELD OF INVENTION

The embodiments described herein relate to methods and apparatuses for generating and/or modifying diagnostic assessments and, in particular, to providing diagnostic assessments using artificial intelligence (AI) based analyses to improve accuracy in delivering diagnoses.

### BACKGROUND

Cancer is the second leading cause of fatality in the United States, causing one in every four deaths. Effective diagnosis and treatment of cancer depend on the ability to correctly diagnose it without undue delay while simultaneously better identifying patients that really need a biopsy versus those who do not. Better outcomes in turn rely on the accuracy and availability of skilled radiologists with the right tools available to them to assist in the diagnostic process.

AI-based systems can be used to assist in diagnostic assessments. Generating AI-based diagnostic assessments and/or integrating the output of AI-based systems into existing clinical workflows, however, can be challenging. Thus, a need exists for systems, devices, and methods to provide improved diagnostic assessments using AI and integrating the output with existing workflows to provide improved diagnostic services.

### SUMMARY

In some embodiments, systems, devices, and methods described herein can assist physicians in performing cancer diagnoses based on medical imaging. Embodiments include a method, comprising receiving, at a first compute device, image data associated with a diagnosis, and an indication of a first diagnostic assessment associated with the image data. The method includes receiving, from a second compute device, an indication of a second diagnostic assessment related to and modified from the indication of the first diagnostic assessment associated with the image data. The method further includes integrating the indication of the second diagnostic assessment with the indication of the first diagnostic assessment to generate an indication of a third diagnostic assessment associated with the image data.

Embodiments disclosed include a method comprising receiving, at a first compute device, image data associated with a region of interest, a first diagnostic assessment associated with the image data, and a second diagnostic assessment associated with the image data, the second diagnostic assessment being different from the first diagnostic assessment. The method includes integrating the second diagnostic assessment with the first diagnostic assessment to generate a third diagnostic assessment associated with the clinical data.

Embodiments disclosed include an apparatus, comprising a memory and a processor operatively coupled to the memory. The processor is configured to receive image data associated with a region of interest, and a first diagnostic assessment associated with the image data. The first diagnostic assessment is in a first format and based on a set of first values assigned to one or more descriptors associated with the image data. The processor is further configured to process the image data using a machine learning (ML) model to generate an output indicating a second diagnostic assessment associated with the clinical data, the second diagnostic assessment in a second format. The processor is further configured to transform the second diagnostic assessment from the second format to the first format. The processor is further configured to generate a third diagnostic assessment by integrating the transformed second diagnostic assessment with the first diagnostic assessment. The transformed second diagnostic assessment is integrated in the form of a set of second values assigned to each descriptor from the one or more descriptors based on the second diagnostic assessment.

Embodiments disclosed include a method comprising receiving, at a compute device, image data associated with a region of interest, and a first diagnostic assessment of the region of interest, the first diagnostic assessment being in a first format. The method includes generating feature vectors associated with the image data, the feature vectors configured to be used to generate a diagnostic assessment of the region of interest associated with the image data. The method includes processing the feature vectors using a machine learning (ML) model to generate an output including a second diagnostic assessment of the region of interest, the second diagnostic assessment being in a second format different from the first format. The method further includes applying a transformation function to the second diagnostic assessment, the transformation function configured to transform the second diagnostic assessment from the first format to the second format. The method further includes determining a third diagnostic assessment of the region of interest based on the applying the transformation function.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The skilled artisan will understand that the drawings primarily are for illustrative purposes and are not intended to limit the scope of the disclosure described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a schematic illustration of an AI-based diagnostic system, according to an embodiment.

FIG. 2 is a schematic representation of a compute device within an AI-based diagnostic system, according to an embodiment.

FIG. 3 is a schematic representation of an AI-based analysis device within an AI-based diagnostic system, according to an embodiment.

FIG. 4 is a flowchart describing a method of integrating a diagnostic assessment in a workflow using an AI-based diagnostic system, according to an embodiment.

FIG. 5 is a flowchart describing a method of generating a diagnostic assessment using an AI-based diagnostic system, according to an embodiment.

FIG. 6 is a flowchart describing a method of generating a clinical decision using an AI-based diagnostic assessment and integrating the AI-based diagnostic assessment within a clinical workflow using an AI-based diagnostic system, according to an embodiment.

FIG. 7 is an example representation of descriptor categories that can be used by an AI-based diagnostic system to generate and/or integrate a diagnostic assessment, according to an embodiment.

FIG. 8 is an example representation of a decision scheme to generate a diagnostic assessment, according to an embodiment.

FIGS. 9A and 9B are schematic representations of example interfaces to provide an improved diagnostic assessment using an AI-based diagnostic system, according to an embodiment.

FIGS. 10A and 10B are schematic representations of example interfaces to provide an improved diagnostic assessment using an AI-based diagnostic system, according to an embodiment.

FIGS. 11A and 11B are schematic representations of example plots showing improved performance of diagnostic assessments generated using an AI-based diagnostic system, according to an embodiment.

### DETAILED DESCRIPTION

Effective diagnosis and treatment of diseases like cancer can depend on an ability to correctly diagnose the illness without undue delay. Correct diagnoses and better outcomes can provide better identification of patients that truly need a biopsy while avoiding unnecessary invasive procedures on patients who may not show indications of needing them. Better outcomes can be based on the accuracy and availability of skilled radiologists with the right tools to assist in the diagnostic process.

The use of AI to provide diagnostic assistance to physicians can be advantageous due to the capability of AI-based systems to learn from clinical data and use that knowledge to render accurate diagnoses. An AI-based diagnostic system can be configured to analyze clinical data and provide a binary output or assign a probability representing the estimated likelihood of a disease condition (e.g., malignancy of a cancer). An AI-based diagnostic system can be configured to improve its diagnostic accuracy with time and exposure to additional data using one or more machine learning tools or models. In some instances, however, appropriately interpreting an output of an AI-based diagnostic system can be significant in determining clinical outcomes.

Additionally, an AI-based diagnostic system can be arbitrarily accurate, and yet have little practical impact if it cannot be effectively utilized by physicians and/or diagnosticians. Integrating the output of AI-based diagnostic systems into existing clinical workflows can be a significant challenge to the widespread adoption of AI-based diagnostic services. Therefore, an effective workflow to integrate output of an AI-based diagnostic system with a conventional system can be an important component along with baseline system accuracy in generating diagnostic assessments.

One obstacle to an effective integration in a workflow can occur, for example, when a physician reads a case, reaches a diagnostic conclusion, and then receives a different recommendation upon consulting an AI-based diagnostic system. For example, the physician can determine a case to be benign therefore not requiring a biopsy, and the AI-based diagnostic system may recommend a biopsy. The physician may then need to reconcile the contradiction, by either disregarding the system output, or accepting the system output and disregarding their own expertise. Neither the physician nor the AI-based system is infallible and forcing the physician to arbitrarily choose one over the other may turn out to be a suboptimal solution to the problem. The AI-based diagnostic systems and methods disclosed herein provide for optimally integrating the diagnostic conclusions drawn by the physician using their existing workflow with the recommendations that may be provided by a decision support system implemented by the AI-based diagnostic system.

There are many lexicon-based reporting systems, which have been proposed and adopted by the medical community, each being in a specified format and/or generated using a specified decision scheme. Several such systems exist for different body parts, such as the Prostate Imaging Reporting and Data System (PI-RADS™), Breast Imaging Reporting and Data System (BI-RADS®), Lung Imaging Reporting and Data System (Lung-RADS), and Liver Imaging Reporting and Data System (LI-RADS™), Colonography Reporting and Data System (C-RADS™), Coronary Artery Disease Reporting and Data System (CAD-RADS™), Neck Imaging Reporting and Data System (NI-RADS™), Ovarian-Adnexal Imaging Reporting and Data System (O-RADS™), and Prostrate Imaging Reporting and Data System (PI-RADS™). The system developed by the American College of Radiology (ACR) for use in diagnosing thyroid nodules in ultrasound is known as American College of Radiology (ACR) Thyroid Imaging Reporting and Data System (TI-RADS™). The American Thyroid Association (ATA) and other groups have developed similar systems with guidelines for assessment and classification of thyroid nodules as well. These systems provide a standardized structure for assessing and reporting on suspicious findings discovered in clinical imaging and for patient management.

Systems, devices, and methods disclosed herein can implement a standardized process to augment and/or adapt diagnostic assessments generated using any number of such reporting systems with AI-based diagnostic assessments, according to some embodiments. In some implementations, systems and methods disclosed can be used to transform diagnostic assessments in one reporting format to another reporting format using any suitable transformation that can be optimized using a suitable performance metric.

In some implementations, the AI-based diagnostic systems disclosed herein can be used for thyroid cancer diagnosis, as described herein. Thyroid nodules are extremely common, presenting in up to 67% of adults in the U.S. on high-resolution ultrasound. The thyroid cancer incidence rate is 14.42 per 100,000 people. Most nodules (˜95%) are benign and many malignant nodules would not result in symptoms or death. Still, over 600,000 fine needle aspirations (FNAs) are performed annually with a positive predictive value of only ˜30%. ACR TI-RADS was developed to standardize diagnostic criteria, reduce biopsy rates, and limit the overdiagnosis of thyroid cancer. TI-RADS increases reader concordance while reducing unnecessary biopsies by 19.9-46.5%. Using the current standard of care, false positive rates and false negative rates remain high. Application of AI-based diagnostic systems as described herein can have a demonstrably positive impact on physicians' decision making, significantly reducing the number of both missed cancers and unnecessary interventional biopsies or fine needle aspirations. (See publications by Ezzat S, et al., entitled “Thyroid incidentalomas-Prevalence by palpation and ultrasonography” in Arch Intern Med. 1994 Aug. 22; by Lim H, et al., entitled “Trends in Thyroid Cancer Incidence and Mortality in the United States” in JAMA. 2017 Apr. 4; by Dean D S et al., entitled “Fine-Needle Aspiration Biopsy of the Thyroid Gland.” updated in 2015 Apr. 26; by Hoang J K et al., entitled “Update on ACR TI-RADS: Successes, Challenges, and Future Directions” in the AJR Special Series on Radiology Reporting and Data Systems. AJR Am J Roentgenol. 2021 March; and by Grani G et al., entitled “Reducing the Number of Unnecessary Thyroid Biopsies While Improving Diagnostic Accuracy: Toward the “Right” TIRADS” in J Clin Endocrinol Metab. 2019 Jan. 1.)

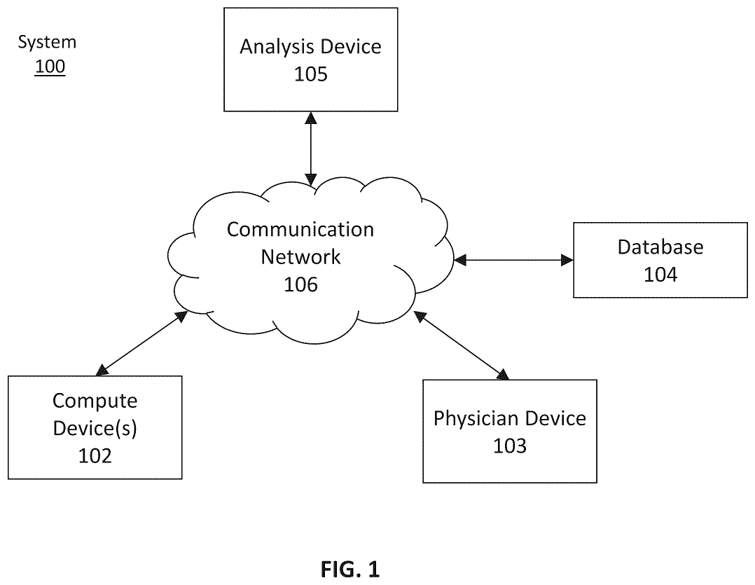

FIG. 1 is a schematic illustration of system 100, which can be an AI-based diagnostic system (“an AID system” or “a system”). The system 100 includes a set of compute devices, including, for example, an analysis device 105 (e.g., AI-based analysis device), a physician device 103, and one or more other compute device(s) 102. The system 100 can aid in delivering and/or augmenting (e.g., adapting, transforming, assessing, etc.) diagnostic assessments provided by users (e.g., physicians, radiologists, diagnosticians, readers, etc.) and/or computer-assisted diagnostic devices. The analysis device 105, physician device 103, and/or compute device(s) 102 can communicate with one another through a communications network 106, as illustrated in FIG. 1.

The analysis device 105 can generate independent diagnostic assessments on clinical data, according to an embodiment. The analysis device 105 can receive diagnostic assessments from one or more users (e.g., physicians, radiologists, diagnosticians, readers, etc.) associated with the set of compute device(s) 102 and/or physician device 103 and augment the diagnostic assessments received from those devices with information based on the independently generated diagnostic assessments. The augmented diagnostic assessments, e.g., adapted, transformed, and/or expanded assessments, can be provided to the compute device(s) 102 and/or physician device 103 via a modified diagnostic assessment, according to an embodiment. In some embodiments, an analysis device 105 can include or be an example of a computer-assisted diagnostic (CAD) device. Suitable examples of CAD devices are provided in U.S. Pat. No. 9,934,567 entitled “Methods and means of CAD system personalization to reduce intra-operator and inter-operator variation,” U.S. Pat. No. 9,536,054 entitled “Method and means of CAD system personalization to provide a confidence level indicator for CAD system recommendations,” and U.S. Pat. No. 10,346,982 entitled “Method and system of computer-aided detection using multiple images from different views of a region of interest to improve detection accuracy,” each of which is incorporated herein by reference in its entirety. In some embodiments, such CAD devices are trained to provide a diagnostic decision or action based on image data of a region of interest of a patient.

In some embodiments, the communication network 106 (also referred to as “the network”) can be any suitable communications network for transferring data, operating over public and/or private networks. For example, the network 106 can include a private network, a Virtual Private Network (VPN), a Multiprotocol Label Switching (MPLS) circuit, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof. In some instances, the communication network 106 can be a wireless network such as, for example, a Wi-Fi or wireless local area network (“WLAN”), a wireless wide area network (“WWAN”), and/or a cellular network. In other instances, the communication network 106 can be a wired network such as, for example, an Ethernet network, a digital subscription line (“DSL”) network, a broadband network, and/or a fiber-optic network. In some instances, the network can use Application Programming Interfaces (APIs) and/or data interchange formats, (e.g., Representational State Transfer (REST), JavaScript Object Notation (JSON), Extensible Markup Language (XML), Simple Object Access Protocol (SOAP), and/or Java Message Service (JMS)). The communications sent via the network 106 can be encrypted or unencrypted. In some instances, the communication network 106 can include multiple networks or subnetworks operatively coupled to one another by, for example, network bridges, routers, switches, gateways and/or the like (not shown).

The compute devices in system 100, including, for example, analysis device 105, physician device 103, and/or other compute device(s) 102 can each be any suitable hardware-based computing device and/or a multimedia device, such as, for example, a server, a desktop compute device, a smartphone, a tablet, a wearable device, a laptop and/or the like. The physician device 103 can be, for example, a workstation or other device such as an ultrasound scanner that is used by a physician, radiologist, diagnostician, reader, or other individual providing care to and/or diagnosing a patient. For example, in some embodiments, the physician device 103 can include a radiology workstation associated with a radiologist and/or a radiological service providing entity. In some instances, the radiology workstation can be equipped to obtain radiology scan data of samples from patients and communicate with the system 100 such that the scans may be transmitted to the analysis device 105, the database 104, and/or other compute device(s) 102. In some instances, the scan data can be suitably updated (e.g., annotated), and/or transmitted from the radiology workstation via a DICOM viewer application. Other compute device(s) 102 can include other devices that are part of an AI-based diagnostic system, including, for example, CAD devices, user, or patient devices, etc.

The system 100 can also include a database 104, e.g., for storing data associated with patients, hospitals, imaging systems, etc. In some instances, the database 104 can include one or more devices that may be a part of a Picture Archival and Communication System (PACS), for example a PACS server. Devices part of a PACS system, such as a PACS server, can be configured for digital storage, transmission, and retrieval of medical images such as radiology images. A PACS server may include software and/or hardware components which directly interface with imaging modalities. The images may be transferred from the PACS server to one or more compute device(s) 102 (e.g., CAD devices) for viewing and/or reporting, and/or to the analysis device 105 for analysis. In some instances, a compute device 102 (e.g., a CAD device) may access images from the database 104 (e.g., a PACS server) and send it to the analysis device 105. In some instances, a compute device 102 (e.g., a CAD device) may obtain images directly from a physician device 103 (e.g., a radiology workstation) and send it to the analysis device 105.

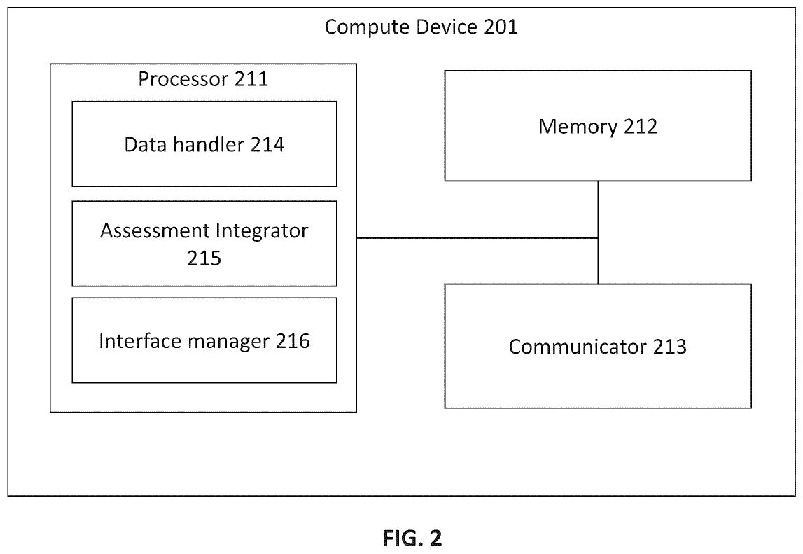

FIG. 2 is a schematic block diagram of an example compute device 201 that can be a part of an AI-based diagnostic system (e.g., system 100), according to an embodiment. The compute device 201 can be structurally and/or functionally similar to the compute device(s) 102 and/or physician device 103 of the system 100 illustrated in FIG. 1. The compute device 201 can be a hardware-based computing device and/or a multimedia device, such as, for example, a server, a desktop compute device, a smartphone, a tablet, a wearable device, a laptop, an ultrasound scanner, and/or the like. The compute device 201 includes a processor 211, a memory 212 (e.g., including data storage), and a communicator 213. In some embodiments, the compute device 201 can have any suitable number of additional components (not shown) including input/output devices, display devices, and the like.

The processor 211 can be, for example, a hardware based integrated circuit (IC), or any other suitable processing device configured to run and/or execute a set of instructions or code. For example, the processor 211 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 211 can be operatively coupled to the memory 212 through a system bus (for example, address bus, data bus and/or control bus).

The processor 211 can be configured to receive and/or obtain clinical data associated with one or more patient(s) and to send that clinical data to an AI-based analysis device (e.g., analysis device 105) in an AI-based diagnostic system. Clinical data can include, for example, image data, histopathological data, medical information, biographic information, information about other details of the patient including health, history, financial status of stability, etc. The processor 211 can be configured to receive and/or obtain clinical data (e.g., image data, medical information, biographic information, other information, etc.) from a remote source (e.g., a clinical database (e.g., database 104), repository, scanner, imaging device, other device associated with a radiological service, devices associated with a patient management system/hospital, devices associated with individuals/patients, etc.) and aid in generating a diagnostic assessment in a predefined format using a predefined decision system or scheme followed by a physician, user, or reader. For example, the processor 211 can be configured to aid a physician in generating a diagnostic assessment using an image reporting and data system (I-RADS) or other image classification system (e.g., the UK 5-point breast imaging scoring system), which can apply to clinical data associated with many parts of the body (for example, breast, thyroid, prostate, etc.), and/or many types of imaging, dependent on the body part in question. In assessing a suspicious finding, a physician using the processor 211 can determine whether an intervention (e.g., follow-up care, biopsy, surgery, etc.) is required, and if so what type of intervention is to be recommended. For example, a breast or thyroid lesion may be left alone if it appears benign. Alternatively, a lesion can be scheduled for a follow-up examination if there is uncertainty. In some circumstances, the lesion can be biopsied and/or excised if it appears suspicious for malignancy. To reach this conclusion, the processor 211 of the compute device 201 can be used (e.g., by a physician and/or CAD system) to evaluate image data to ascertain a risk assessment.

In some instances, the processor 211 of the compute device 201 can be configured to generate the risk assessment based on consideration of a list of image descriptors, each of which can be indicative of a positive or negative prognosis. The descriptors can be specified by the processor 211 according to an I-RADS or other image classification system or decision scheme and can be different for different body parts, anatomies, and/or modalities. Using BI-RADS on a breast ultrasound, for example, a physician can give assessments of a lesion based on descriptors including, for example, shape, orientation, echogenicity, margins, and posterior acoustic effects. The processor 211 can be configured to implement the image classification system to specify how to interpret a chosen set of descriptors and determine what type of intervention is merited by the finding. Some of these image classification systems can be point-based, and some others can be rule-based. The processor 211 can also be configured to maintain a log of clinical information related to the clinical data (e.g., name or other identifier of the patient, medical history, time, and date of receiving or generating the clinical data and/or the diagnostic decision, timeline of recommended intervention, etc.). In some instances, the decision for intervention, the degree of intervention, and/or the timing of intervention can depend on factors such as health or prior history of the patient, the interest of the patient to undergo the procedure, the level of comfort of the patient to undergo the procedure, cost considerations or personal circumstances of the patient, other costs associated with the procedure, a projected trajectory of medical care available, and/or the like. The processor 211 of the compute device 201 can be further configured to generate and/or adjust the risk assessment based on one or more of these factors.

The processor 211 can include or execute one or more module(s) or instruction(s) stored in memory 212 to function as a data handler 214, an assessment integrator 215, and an interface manager 216. In some embodiments, the processor 211 can include software applications (not shown in FIG. 2) that can be used to provide an interface to deliver diagnostic assessments. The software application can be any suitable software or code that when executed by the processor 211 can be configured to perform a group of coordinated functions, tasks, or activities for the benefit of a user of the compute device 201. Software application can be, for example, browser applications, word processing applications, media playing applications, JAVA based applications, image rendering or editing applications, text editing applications, and/or the like.

In some embodiments, each of the data handler 214, the assessment integrator 215, the interface manager 216, and/or the software applications can be software stored in the memory 212 and executed by the processor 211. For example, each of the above-mentioned portions of the processor 211 can be implemented in the form of instructions that can include code that is configured to cause the processor 211 to execute the data handler 214, the assessment integrator 215, the interface manager 216, and/or the software applications. The code can be stored in the memory 212 and/or a hardware-based device such as, for example, an ASIC, an FPGA, a CPLD, a PLA, a PLC and/or the like. In other embodiments, each of the data handler 214, the assessment integrator 215, the interface manager 216, and/or the software applications can be hardware configured to perform their respective functions.

The processor 211 implementing data handler 214 can be configured to collect clinical data related to patients and/or assessments conducted, and the software applications executed in the compute device 201. For example, the data handler 214 can receive, collect, processor, and/or store image data such as radiological data provided for an assessment, clinical information related to radiological data provided for assessment, and/or other information associated with a patient undergoing a cancer assessment. In some embodiments, the processor 211 can implement data handler 214 as a background process that monitors for newly incoming data, processes that data, and/or stores that data.

In some implementations, the processor 211 implementing the data handler 214 can be configured to aid a physician in generating a diagnostic assessment according to a specified image classification system or decision scheme (e.g., BI-RADS, TI-RADS, Lung-RADS, etc.). For example, the processor 211 can be configured to cause an input/output device (e.g., an input/output device coupled to communicator 213 or integrated into compute device 201) to display image data to a physician.

The processor 211 implementing assessment integrator 215 can be configured to receive a first diagnostic assessment (or a signal or other indication representative of a first diagnostic assessment) of a region of interest associated with a set of image data. As an example, the first diagnostic assessment can be a point-value based (e.g., 1-5) or a rule-based indication of a degree of malignancy of a cancer or cancerous tissue. For example, the assessment integrator 215 can receive the first diagnostic assessment from the data handler 214 having implemented a decision scheme as directed by a physician. In some embodiments, the first diagnostic assessment can be an assessment made by a physician viewing, for example, image data associated with the region of interest, a patient's history, or other information of a patient. As another example, the assessment integrator 215 can receive the first diagnostic assessment via the communicator 213, e.g., from a remote source. The first diagnostic assessment can be in a first format (e.g., according to a first decision scheme such as, for example, BI-RADS, TI-RADS, Lung-RADS, etc.).

The assessment integrator 215 can be configured to receive a second diagnostic assessment (or a signal or other indication representative of a second diagnostic assessment) associated with the same set of image data and/or a different set of image data of the same region of interest. For example, the second diagnostic assessment can be an AI-based diagnostic assessment provided by an analysis device such as a CAD device (e.g., similar to the analysis device 105 of system 100 in FIG. 1). The assessment integrator 215 can be configured to integrate the first diagnostic assessment with the second diagnostic assessment to generate a third diagnostic assessment that can provide a user, via the compute device 201, a modified, adapted or more comprehensive diagnostic assessment via an interface. The modified, adapted or more comprehensive diagnostic assessment can combine the information from the first diagnostic assessment and the second diagnostic assessment to be easily interpreted and/or used by a physician or other.

In some embodiments, the assessment integrator 215 can perform the integration of the first diagnostic assessment with the second diagnostic assessment following a transformation of the second diagnostic assessment, as described herein. In some instances, the assessment integrator 215 can receive the second diagnostic assessment in a transformed form (e.g., receive from an analysis device such as the analysis device 105 and/or 305), and perform the integration of the first diagnostic assessment with the second diagnostic assessment. In some instances, the integration can be by combining two or more diagnostic assessments in a point-value format.

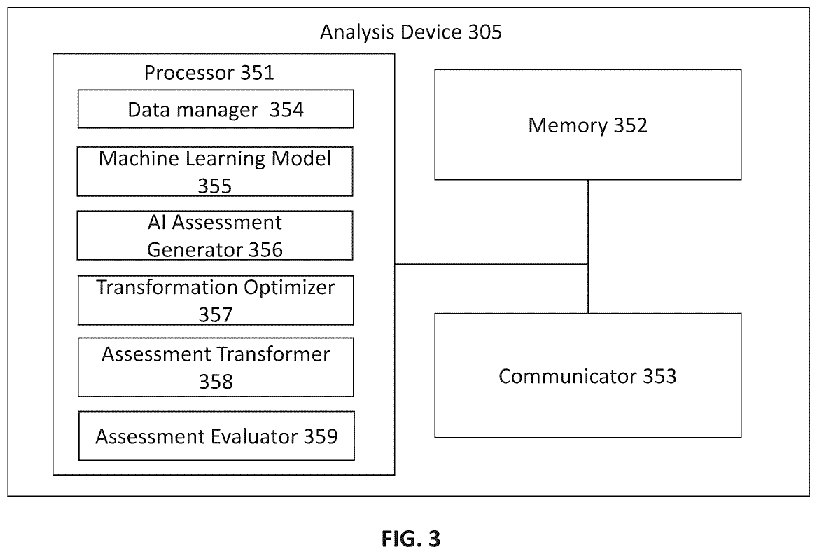

While described herein as performed by a compute device 201 that can be separate from an analysis device that provides an AI-based diagnostic assessment (e.g., a CAD device), the compute device 201 implementing the assessment integrator 215, in some embodiments, can be the same analysis device that provides the AI-based diagnostic assessment. In other words, one or more of the above-described processes described with respect to the processor 211, e.g., implementing the assessment integrator 215, can be performed by an analysis device (e.g., analysis device 105 that is included in the system 100 shown in FIG. 1, and/or the analysis device 305 shown in FIG. 3 and described in further detail in the following sections).

The interface manger 216 can be configured to provide control options to a user, via an interface (also referred to herein as “user interface”), for example, to modify and/or customize settings in an interface to provide diagnostic assessment to a physician. In some implementations, the interface manager 216 can be configured to allow a switchable selection of display of the integrated modified, adapted and/or more comprehensive diagnostic assessment based on a user preference. In some instances, the interface manger 216 can be configured to modify a user interface from displaying a first diagnostic assessment (e.g., a physician generated diagnostic assessment) to display a third diagnostic assessment that is based on an integration of the first diagnostic assessment (physician generated diagnostic assessment) and the second diagnostic assessment (AI-based diagnostic assessment), as described herein.

While the compute device 201 is described to implement, via the processor 211, each of a data handler, an assessment integrator, and an interface manager, it can be appreciated that a compute device can be configured with several instances of the above-mentioned units, components, and/or modules. Moreover, the terms data handler, assessment integrator, and interface manager are provided for illustrative purposes, e.g., to explain the processes implemented by the processor 211. Therefore, one or more of these modules can be combined into single modules or generally referred to as a processor configured to perform one or more processes or steps thereof.

The memory 212 of the compute device 201 can be, for example, a random-access memory (RAM), a memory buffer, a hard drive, a read-only memory (ROM), an erasable programmable read-only memory (EPROM), and/or the like. The memory 212 can store, for example, one or more software modules and/or code that can include instructions to cause the processor 211 to perform one or more processes, functions, and/or the like (e.g., the data handler 214, the assessment integrator 215, the interface manger 216, and/or the software applications described above). In some embodiments, the memory 212 can include extendable storage units that can be added and used incrementally. In some implementations, the memory 212 can be a portable memory (for example, a flash drive, a portable hard disk, and/or the like) that can be operatively coupled to the processor 211. In other instances, the memory can be remotely operatively coupled with the compute device. For example, a remote database server can serve as a memory and be operatively coupled to the compute device.

The communicator 213 can be a hardware device operatively coupled to the processor 211 and memory 212 and/or software stored in the memory 212 executed by the processor 211. The communicator 213 can be, for example, a network interface card (NIC), a Wi-Fi™ module, a Bluetooth® module and/or any other suitable wired and/or wireless communication device. Furthermore, the communicator 213 can include a switch, a router, a hub and/or any other network device. The communicator 213 can be configured to connect the compute device 201 to a communication network (such as the communication network 106 shown in FIG. 1). In some instances, the communicator 213 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the communicator 213 can facilitate receiving and/or transmitting a file and/or a set of files through a communication network (e.g., the communication network 106 in the system 100 of FIG. 1). In some instances, a received file can be processed by the processor 211 and/or stored in the memory 212 as described in further detail herein. In some instances, as described previously, the communicator 213 can be configured to send data collected and/or analyzed by the data handler 214 to an analysis device (e.g., analysis device 105) of an AI-based diagnostic system (e.g., AI-based diagnostic system 100) to which the compute device 201 is connected. The communicator 213 can also be configured to send data collected and analyzed by the data handler 215 and the results of any analyses generated by the assessment integrator 215 and/or the interface manager 216, to the analysis device of an AI-based diagnostic system to which the compute device 201 is connected.

Returning to FIG. 1, the compute device 102 that is connected to the system 100 can be configured to communicate with the analysis device 105 via the communication network 106. FIG. 3 is a schematic representation of an analysis device 305 that is part of an AI-based diagnostic system (e.g., AI-based diagnostic system 100), according to embodiments. The analysis device 305 can be structurally and/or functionally similar to the analysis device 105 of the system 100 illustrated in FIG. 1. The analysis device 305 includes a communicator 353, a memory 352, and a processor 351.

Similar to the communicator 213 within compute device 201 of FIG. 2, the communicator 353 can be a hardware device operatively coupled to the processor 351 and the memory 352 and/or software stored in the memory 352 executed by the processor 351. The communicator 353 can be, for example, a network interface card (NIC), a Wi-Fi™ module, a Bluetooth® module and/or any other suitable wired and/or wireless communication device. Furthermore, the communicator 353 can include a switch, a router, a hub and/or any other network device. The communicator 353 can be configured to connect the analysis device 305 to a communication network (such as the communication network 106 shown in FIG. 1). In some instances, the communicator 353 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

The memory 352 can be a random-access memory (RAM), a memory buffer, a hard drive, a read-only memory (ROM), an erasable programmable read-only memory (EPROM), and/or the like. The memory 352 can store, for example, one or more software modules and/or code that can include instructions to cause the processor 351 to perform one or more processes, functions, and/or the like. In some implementations, the memory 352 can be a portable memory (e.g., a flash drive, a portable hard disk, and/or the like) that can be operatively coupled to the processor 351. In other instances, the memory 352 can be remotely operatively coupled with the analysis device 305. For example, the memory can be a remote database server operatively coupled to the analysis device 305 and its components and/or modules.

The processor 351 can be a hardware based integrated circuit (IC) or any other suitable processing device configured to run and/or execute a set of instructions or code. For example, the processor 351 can be a general-purpose processor, a central processing unit (CPU), a graphics processing unit (GPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 351 is operatively coupled to the memory 352 through a system bus (e.g., address bus, data bus and/or control bus). The processor 351 is operatively coupled with the communicator 353 through a suitable connection or device as described in further detail.

The processor 351 can be configured to include and/or execute several components, units and/or modules that may be configured to perform several functions, as described in further detail herein. The components can be hardware-based components (e.g., an integrated circuit (IC) or any other suitable processing device configured to run and/or execute a set of instructions or code) or software-based components (executed by the server processor 352), or a combination of the two.

As illustrated in FIG. 3, the processor 351 includes or can execute one or more module(s) or instruction(s) stored in the memory 352 to function as a data manager 354, a machine learning model 355, an AI assessment generator 356, a transformation optimizer 357, an assessment transformer 358, and an assessment evaluator 359.

The data manager 354 in the processor 351 can be configured to receive communications between the analysis device 305 and compute devices connected to the analysis device 305 through suitable communication networks (e.g., compute devices 101-103 connected to the analysis device 105 via the communication network 106 in the system 100 in FIG. 1). The data manager 354 is configured to receive, from the compute devices, and/or from remote sources, information pertaining to diagnostic assessments, clinical decisions, patient identification, etc.

The data manager 354 can be configured to receive radiological imaging data from patients the data being associated with organs and/or organ systems under clinical or therapeutic analysis or study. In some instances, the data manager 354 can receive information associated with medical history of patients, treatments provided, invasive procedures undergone, etc. The data manager 354 can receive ground truth data or labeled data that can be used as training data to train the Machine Learning (ML) model 355 to generate AI-based diagnostic assessments with high accuracy. The imaging data can be in the form of DICOM images and associated meta-data, or other imaging data.

The ML model 355 can be implemented to generate an AI-based diagnostic assessment based on image data. Models may include, but are not limited to, deep neural networks, multi-layer perceptrons, random forests, support vector machines, etc.

The processor 351 includes an AI assessment generator 356, implemented to generate an AI-based diagnostic assessment of image data using the ML model 355. The AI assessment generator 356 can be configured to receive image data and generate feature vectors that can be provided to the ML model 355 to generate the AI-based diagnostic. In some instances, for example, the ML model 355 can be configured to output a risk estimate that can be associated with a likelihood of malignancy of a nodule captured in the image data.

The processor 351 includes a transformation optimizer 357 that is configured to receive a first diagnostic assessment in a first format and a second diagnostic assessment (or a signal or other indication representative of a second diagnostic assessment) in a second format that is different from the first format. The transformation optimizer 357 can be configured to generate a parameterized transformation function that can transform or map the second diagnostic assessment (or a signal or other indication representative of the second diagnostic assessment) in the second format to the first format of the first diagnostic assessment such that the second diagnostic assessment can be integrated with the first diagnostic assessment to generate a third diagnostic assessment.

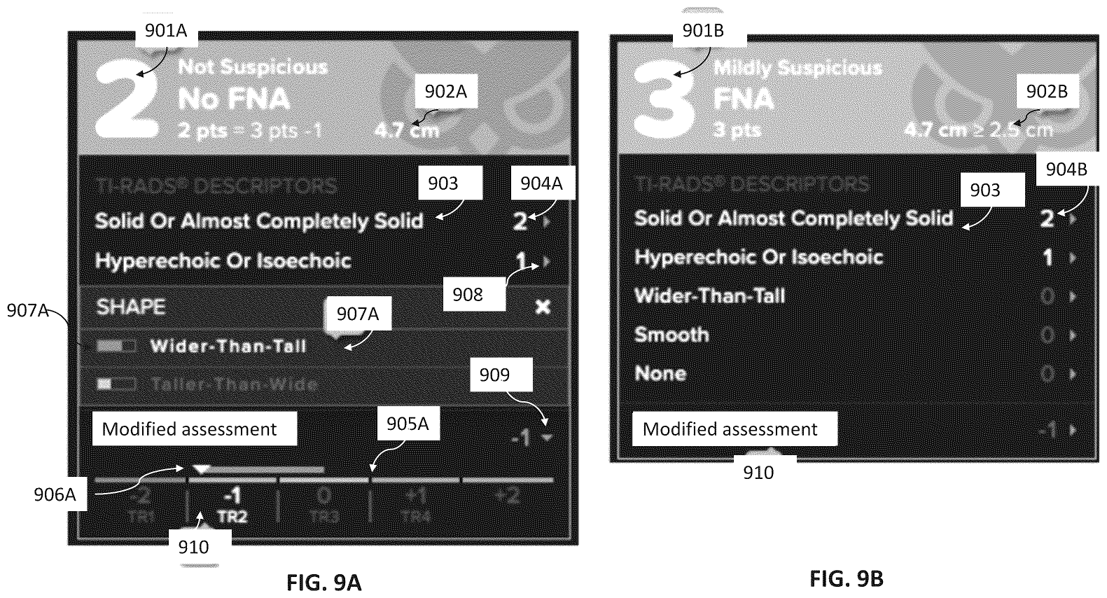

The transformation optimizer 357 can be configured to identify the parameters such that the transformation function can be used to map or transform a second diagnostic assessment, for example an AI-based diagnostic assessment (in a format of an output by an ML model or compatible with the output of the ML model 355), to a format of a first diagnostic assessment (or a signal or other indication representative of a first diagnostic assessment) in a first format, for example a physician derived diagnostic assessment in a format that is based on a standardized or established point-based or rule-based decision scheme such as TI-RADS, BI-RADS, Lung-RADS, etc. As an example, in some instances, the transformation optimizer 357 can be configured to identify parameters such that a transformation function can be used to map or transform an AI-based diagnostic assessment in the form of a set of probabilities associated with classes defined according to a predetermined classification system into an integer point value that is compatible with a physician-based diagnostic assessment. For example, the transformation optimizer 357 can transform probabilities that are output by an AI-based ML model (e.g., ML model 355) that is trained using a classification system distinguishing classes defining malignancy of cancers based on various features, conditions, or factors. The transformation optimizer 357 can map these probabilities on to a scale based on an integer point-value (e.g., a point value system associated with a descriptor used to generate a standardized or established classification system (e.g., TI-RADS, BI-RADS, Lung-RADS, etc.). In some implementations, the transformation optimizer 357 can identify set of descriptors with a first set of point values (or integer values) on a predetermined scale, and an overall score based on the descriptors/point values associated with a first diagnostic assessment in a format according to a standardized classification system. Each descriptor from the set of descriptors can be associated with a first point value from the first set of point values mapped on the predetermined scale. The transformation optimizer 357 can be configured to transform a second diagnostic assessment, which can be in the form of probabilities output by an AI-based ML model, such that it provides a second set of point values (or integer values) each point value in the second set of point values being associated with one descriptor from the set of descriptors, and each point value in the second set of point values being mapped on the predetermined scale. Each point value from the second set of point values that is associated with each descriptor can correspond to or be configured to adjust or update a counterpart point value from the first set of point values associated with that descriptor in the first diagnostic assessment that is in the format according to the standardized classification system. Said in another way, the transformation optimizer 357 can identify the descriptors and first point values of each descriptor associated with the first diagnostic format and optimize the transformation function to map the output of an ML model (e.g., in the form of probabilities) to provide second point values of each descriptor, on a predetermined scale associated with the first diagnostic format, such that the second point values can be integrated with the first point values to generate a third diagnostic assessment (e.g., FIG. 9A,).

In some instances, the transformation optimizer 357 can be configured to transform a second diagnostic assessment, which can be in the form of probabilities output by an AI-based ML model, such that it provides a single point value configured to update or adjust the overall score associated with the first diagnostic assessment or the physician-based diagnostic assessment (instead of a point value adjustment for each descriptor). In some instances, the transformation optimizer 357 can be configured to transform a second diagnostic assessment, which can be in the form of probabilities output by an AI-based ML model, such that it provides an overall modified risk assessment that can be tied to not just medical or clinical information but also other related factors such as costs and/or risks associated with performing a recommended clinical action.

In some instances, the transformation optimizer 357 can be configured to optimize the transformation function using any suitable method to generate an optimized transformation function that maximizes a specified performance of the transformation function according to a specified metric used to evaluate assessments. In some instances, the transformation optimizer 357 can implement an objective function that is used to optimize the transformation function based on predefined criteria. For example, the transformation optimizer 357 can be configured to optimize the transformation function to maximize the positive impact of integrating an AI-based diagnostic assessment with a physician based diagnostic assessment in terms of a specified metric. The specified metric can be any suitable metric including metrics like area under the curve (AUC), statistics associated with Receiver Operating Characteristics (ROC) curves, sensitivity, and specificity. In some instances, the transformation optimizer 357 can be configured to generate and/or optimize a parameterized transformation function to integrate one AI-based diagnostic assessment in a first format with another AI-based diagnostic assessment in a second format to generate an integrated diagnostic assessment. In some instances, the transformation optimizer 357 can be configured to generate and/or optimize a parameterized transformation function to integrate one physician-based diagnostic assessment in a first format with another physician-based diagnostic assessment in a second format to generate an integrated diagnostic assessment.

The processor 351 includes an assessment transformer 358 that is configured to apply the transformation function generated by the transformation optimizer 357. In some instances, the assessment transformer 358 can also be configured to integrate the transformed second diagnostic assessment (e.g., AI-based diagnostic assessment) or the output of the transformation with the first diagnostic assessment (e.g., physician-based diagnostic assessment) to generate a third diagnostic assessment that combines information from two indications of diagnostic assessments which may be more or less aligned in the clinical decision that is recommended by each diagnostic assessment.

The processor 351 includes an assessment evaluator 359 that is configured to evaluate a performance of the diagnostic assessments generated using the AI-based system, the physician-based system, and/or the modified assessment generated by integrating two or more diagnostic assessments. The assessment evaluator 359 can be configured to perform the evaluation based on predefined criteria including metrics like AUC, ROC statistics, sensitivity, specificity, etc. The assessment evaluator 359 can be configured to generate analytical data associated with a performance the physician-based diagnostic assessments, and performance of modified diagnostic assessments integrating physician-based and AI-based diagnostic assessment using ground truth data or data including confirmation or correction via true findings of cancerous nature of tissue (e.g., via biopsies/FNAs). The assessment evaluator 359 can be configured to provide feedback to the AI-based system and/or the transformation optimizer 357 and/or the assessment transformer 358 to further improve performance of the AI-based system and/or the modified diagnostic assessments.

While the analysis device 305 is described to implement, via the processor 351, each of a data manager, an ML model, an AI assessment generator, an assessment transformer, an assessment modifier, and an assessment evaluator, in other embodiments, an analysis device similar to the analysis device 305 can be configured with several instances of the above-mentioned units, components, and/or modules. For example, in some embodiments, the server may include several data managers, several AI assessment generators, several assessment transformers, several assessment modifiers and/or several ML models associated with one or more compute devices or groups of compute devices. Moreover, the terms data manager, ML model, AI assessment generator, assessment transformer, assessment modifier, and assessment evaluator are provided for illustrative purposes, e.g., to explain the processes implemented by the processor 351. Therefore, one or more of these modules can be combined into single modules or generally referred to as a processor configured to perform one or more processes or steps thereof. In addition, while the analysis device 305 is presented as a separate device from the compute device 201 but in communication in such compute device 201, it can be appreciated that the analysis device 305 and the compute device 201 (or other analysis devices and/or compute devices described herein) can be implemented on one or more devices that include suitable components (e.g., processors, memories, etc.) for performing the processes described with reference to each.

While the analysis device 305 is described herein to have a data manager, an ML model, an AI assessment generator, a transformation optimizer, an assessment transformer, and an assessment evaluator, in other embodiments an analysis device similar in structure and/or function to the analysis device 305 can be configured such that portions of the above described functions and/or modules can be carried out in and/or executed by compute devices that are included in the system (e.g., compute device 201) for example, via client side applications installed in the compute devices (e.g., within the data handler 214 of FIG. 2). Similarly stated, in some instances, functions described as being performed on an analysis device (e.g., analysis device 305) can be performed on a compute device 201 and vice versa.

In use, a compute device (e.g., compute device 201 or any of the other compute devices described herein) in an AI-based diagnostic system as described herein can receive image data associated with a region (e.g., tissue or organ) of interest of a patient. In some embodiments, the compute device can implement a standardized decision scheme to generate a first diagnostic assessment (or a signal or other indication representative of a diagnostic assessment) associated with the image data. Alternatively, the compute device can receive a first diagnostic assessment from a physician, e.g., viewing the image data. The compute device can then send information associated with the image data or send the image data to an analysis device in the AI-based diagnostic system. In some instances, the compute device can also send information indicating the standardized format or decision scheme (e.g., BI-RADS, TI-RADS, Lung-RADS, etc.) that is being used to generate the first diagnostic assessment. The analysis device can receive the image data and generate a second diagnostic assessment or AI-based diagnostic assessment (or a signal or other indication representative of such a diagnostic assessment) associated with the image data (e.g., using an ML model). The analysis device can further generate a transformation function that is configured to map or transform the second AI-based diagnostic assessment to the format of the first diagnostic assessment. The analysis device can optimize the transformation function such that the transformation of the second AI-based diagnostic assessment to the format of the first diagnostic assessment is configured to achieve a target of maximizing a specified performance metric of the transformed diagnostic assessment. The analysis device can then apply the optimized transformation function to the second AI-based diagnostic assessment to compute a modified third diagnostic assessment (or a signal or other indication representative of a diagnostic assessment) integrating the first and the second diagnostic assessments. The analysis device can then send the modified third diagnostic assessment to the compute device. The compute device can integrate the modified third diagnostic assessment with the first diagnostic assessment and present the integrated assessment and/or a recommended intervention to a user via an interface.

In some embodiments, AI-based diagnostic systems described herein (or components of such systems, such as any of the compute devices described herein) can be configured to work with any imaging, reporting, and data system, including any suitable I-RADS (e.g., TI-RADS, BI-RADS, lung-RADS, etc.) or any other standardized and/or lexicon-based classification and reporting system to help physicians more accurately classify suspicious lesions or nodules. Some such systems can include or implement a machine learning model, or an AI-based engine associated with the machine learning model for risk assessment of medical image data. Such systems can suitably generate outputs of the AI-based engine in an overall modified diagnostic assessment to positively impact clinical performance and/or patient management. The machine learning model and/or the AI-based engine can be tuned to adjust operating points to promote desired properties in generating assessments or modified assessments. For example, such systems can be tuned to achieve greater sensitivity and/or specificity in diagnostic assessments compared to a ground truth data set depending on desired output goals. The output of the AI-based diagnostic systems may differ depending on the type of standardized, lexicon-based classification and reporting system used by the physician or technician and may include an adjusted integer scale score used to adjust the overall output score of the classification system, and/or a risk percentage output that shifts the score of a lesion or nodule across classification categories based on the AI analysis of the image data, or a similar scale adjustment.

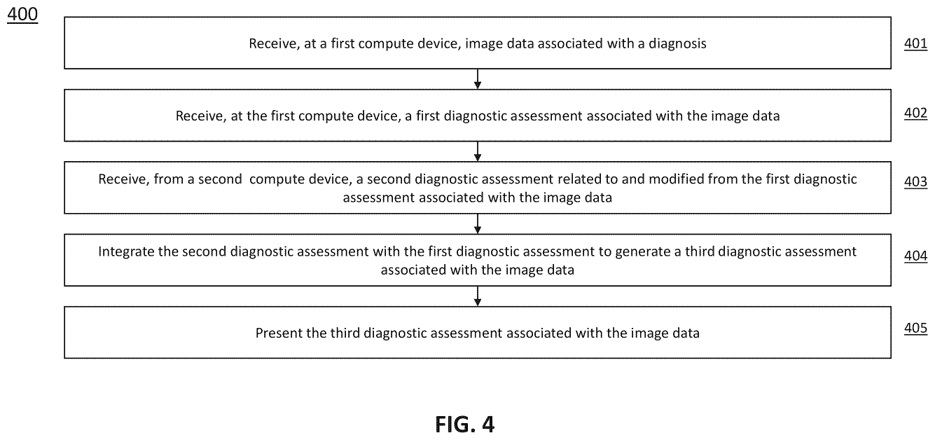

FIG. 4 illustrates a flowchart describing a method of generating and presenting a modified diagnostic assessment using an AI-based diagnostic system, according to an embodiment. The method 400 can be implemented by a compute device that is similar in structure and/or function to the compute devices 102, 201 and/or analysis devices 105, 305 described above.

The method 400 at 401 includes receiving, at a first compute device, image data associated with a diagnosis. The image data can be radiological imaging data associated with a lesion or nodule in an organ or an organ system of a patient. At 402, the method 400 incudes receiving, at the first compute device, a first diagnostic assessment (or a signal or other indication representative of a first diagnostic assessment) associated with the image data. In some implementations, the first diagnostic assessment can be a physician-based assessment using a standardized decision scheme and/or classification or reporting system including, for example, a system such as BI-RADS, TI-RADS, Lung-RADS, etc. The first diagnostic assessment can include a set of descriptors based on the standardized classification or reporting system with each descriptor being associated with a point value, and an overall score based on the point values associated with the descriptors, the overall score being used to make a clinical recommendation according to a standardized decision scheme. The decision scheme can be based on a cumulative point-value and/or rule-based systems, for example, a first decision based on the overall score meeting a first criterion associated with a first threshold value, and the like.

At 403, the method 400 includes receiving, from a second compute device, a second diagnostic assessment (or a signal or other indication representative of a second diagnostic assessment) related to and modified from the first diagnostic assessment associated with the image data. The second diagnostic assessment can be an AI-based diagnostic assessment. In some instances, the second compute device can be an analysis device similar in structure and/or function to the analysis device 105 and/or 305 described above. In some embodiments, the first and second compute devices as described herein can be the same device, and in such embodiments, the method 400 at 403 can obtain a second diagnostic assessment by using a trained machine learning model to process the image data and obtain a second diagnostic assessment or AI-based diagnostic assessment.

In some implementations, the second diagnostic assessment can be in the form of an output of an ML model. In some implementations, the second diagnostic assessment can be in the form of one or more probabilities or likelihood of the image data indicating or including features indicating an assignment of the image data to one or more predefined classes (e.g., malignant, benign, etc.).

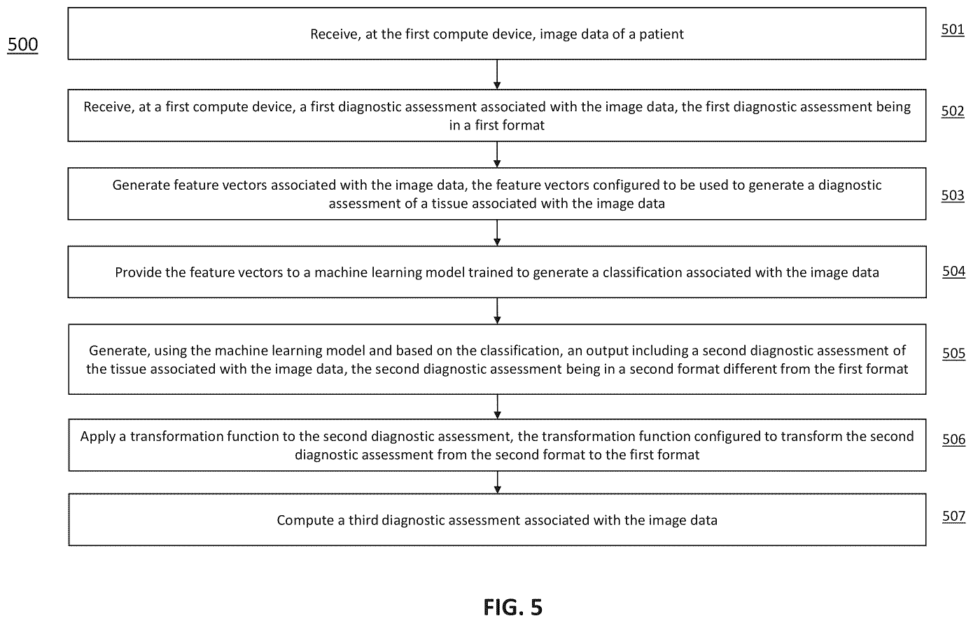

At 404, the method 400 includes integrating the second diagnostic assessment with the first diagnostic assessment to generate a third diagnostic assessment (or a signal or other indication representative of a third diagnostic assessment) associated with the image data. In some embodiments, the second diagnostic assessment can first be transformed to be compatible with the format of the first diagnostic system, for example as explained with reference to the method 500 in FIG. 5. The transformed second diagnostic assessment can then be integrated with the first diagnostic assessment to generate the third diagnostic assessment. In some implementations, the second diagnostic assessment can be integrated with the first diagnostic assessment through indications of adjustments or modifications made based on one or more descriptors associated with the first diagnostic assessment. In some implementations, the second diagnostic assessment can be integrated with the first diagnostic assessment through indications of adjustments or modifications made based on the overall score associated with the first diagnostic assessment.

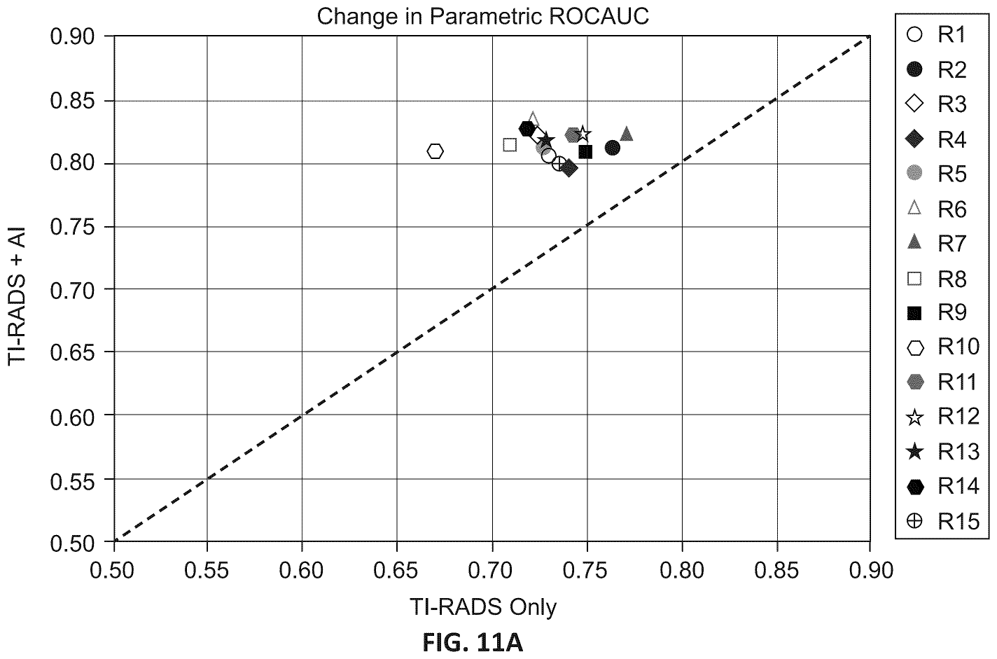

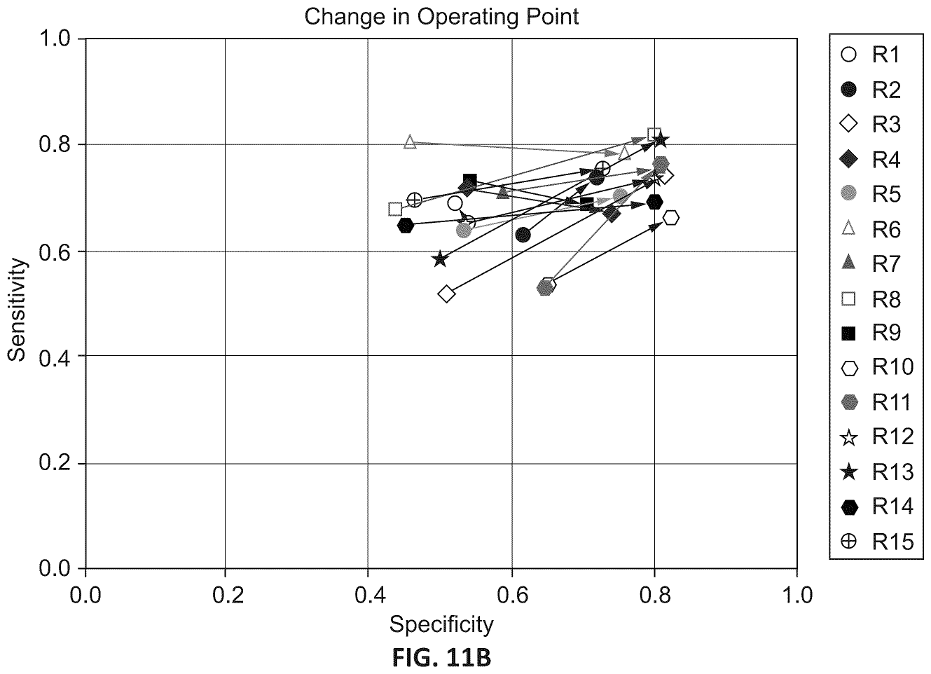

At 405, the method includes presenting the third diagnostic assessment associated with the image data to a user via an interface. FIGS. 9A-9B and 11A-11B described in further detail below illustrate example interfaces that present example diagnostic assessments (or a signal or other indication representative of diagnostic assessments).

FIG. 5 illustrates a flowchart describing a method of generating a diagnostic assessment using an AI-based diagnostic system, according to an embodiment. The method 500 can be implemented by a compute device that is similar in structure and/or function to the compute devices 102, 201 and/or analysis devices 105, 305 described above.

At 501, the method 500 includes receiving, at a first compute device, image data associated with a region (e.g., tissue or organ) of interest of a patient.

At 502, the method includes receiving, at the first compute device, a first diagnostic assessment associated with the image data, the first diagnostic assessment being in a first format. In some instances, the first diagnostic assessment can be a physician-based assessment and the format can be according to a standardized classification or reporting system (e.g., TI-RADS, BI-RADS, Lung-RADS, etc.) associated with a standardized decision scheme to make clinical recommendations. In some implementations, the first diagnostic assessment can include a set of descriptors, each descriptor from the set of descriptors being associated with a first point value, and the first point value associated with all descriptors collectively forming a first set of point values according to an established or standardized classification or reporting system (e.g., TI-RADS, BI-RADS, Lung-RADS, etc.). The first diagnostic assessment can further include a first overall score based on the first set of point values (e.g., an overall score generated from cumulative combination of the first set of point values). The first overall score can be on a predetermined scale and configured to indicate a degree of severity of the cancerous nature of the region (tissue or organ) of interest based on the value and its location on the predetermined scale. The first overall score can be used to provide a first clinical recommendation based on an established rule-based decision scheme.

At 503, the method 500 includes generating feature vectors associated with the image data, the feature vectors configured to be used to generate a diagnostic assessment of the region (e.g., tissue or organ) of interest associated with the image data.

At 504, the method 500 includes providing the feature vectors to a ML model trained to generate a classification associated with the image data. The ML model can be similar in structure and/or function to the ML model 355 described previously.

At 505, the method 500 includes generating, using the machine learning model, and based on the classification, an output including a second diagnostic assessment of the region (e.g., tissue or organ) of interest associated with the image data, the second diagnostic assessment being in a second format different from the first format. In some implementations, the second diagnostic assessment can be in a second format that is in the form of probabilities or likelihood that the image data includes features indicating that the region (e.g., tissue or organ) of interest falls under identified classes defined by the classification learned during training by the ML model. The classification system used by the ML model can be any suitable classification system including any suitable definition of classes using any feature or characteristic associated with the image data and/or region (e.g., tissue or organ) of interest, and/or established standards of evaluation. As an example, the identified classes can be malignant and benign. As another example, the identified classes can include classes defined based on varying degrees of malignancy, and/or the like.

At 506, the method 500 includes applying a transformation function to the second diagnostic assessment, the transformation function configured to transform the second diagnostic assessment from the second format to the first format. In some instances, the transformation function can be generated using predefined parameters selected to map diagnostic assessments in the second format to the first format in a suitable manner. In some instances, the transformation function can be optimized using an objective function such that the transformation of diagnostic assessments from the second format to the first format meets specified performance criteria (e.g., AUC, ROC statistics, sensitivity, specificity of diagnostic assessment, etc.).

In some implementations, the second diagnostic assessment can include a set of probabilities provided by an output of a ML model the probabilities indicating the likelihood of an image data including features indicating that the region (tissue or organ) featured in the image data belongs to one or more identified classes. The transformation function can be optimized and configured to transform the second diagnostic assessment by mapping the probabilities onto a predetermined integer or point value scale that is compatible with the first diagnostic assessment. In some implementations the transformation function can be configured to map each probability from a plurality of probabilities to generate a second set of point values, each point value from the second set of point values being associated with

In some implementations, the applying a transformation function to the second diagnostic assessment can be directed to generate a second set of point values based on the probabilities such that each descriptor from the set of descriptors in the first diagnostic assessment is associated with a second point value from the second set of point values that are based on the second diagnostic assessment. Said in another way, the transformation function can be used to generate a second set of point values based on the second diagnostic assessment such that each descriptor from the set of descriptors in the first diagnostic assessment is associated with a first point value based on the first diagnostic assessment and a second point value based on the transformed second diagnostic assessment. In some instances, the second diagnostic assessment can include a confidence level indicator (CLI) associated with each second point value based on the second diagnostic assessment.

In some implementations, the applying a transformation function to the second diagnostic assessment can be directed to generate a second overall score based on the probabilities, the second overall score configured to indicate a degree of severity of the cancerous nature of the region (tissue or organ) of interest in the image data and based on the second diagnostic assessment. The second overall score can be mapped on the same predetermined scale as the first overall score and can be integrated with the first overall score to provide an adjustment or modification to the first overall score. In some instances, the second diagnostic assessment can include a confidence level indicator (CLI) associated with the second overall score.

At 507, the method includes computing a modified third diagnostic assessment associated with the image data, based on the transformation of the second diagnostic assessment from the second format to the first format, and based on integrating the transformed second diagnostic assessment and the first diagnostic assessment. In some implementations, the transformation can be in the form of generation of a second set of point values based on the second diagnostic assessment. In some implementations, the integration can be in the form of integration of the first point value (based on the first diagnostic assessment) associated with each descriptor from the set of descriptors with the second point value (based on the transformed second diagnostic assessment) associated with that descriptor to generate the third diagnostic assessment including a third point value associated with that descriptor. The third diagnostic assessment can include a third overall score based on all the third point values associated with the set of descriptors. The third overall score can be mapped on the same predetermined scale as the first overall score and can be used to make an updated clinical recommendation according to the standardized decision scheme. As described previously, the decision scheme can be based on a cumulative point-value and/or rule-based systems, for example, a first decision based on the third overall score meeting the first criterion associated with the first threshold value, and the like.

In some implementations, the transformation can be in the form of generation of a second overall score mapped on to the predetermined scale of the first overall score and associated with the second diagnostic assessment (rather than a set of second point values based on the second diagnostic assessment). The computing the third diagnostic assessment associated with the image data, can be based on integration of the first overall score with the second overall score to generate a third overall score. The third overall score can be mapped on the same predetermined scale as the first overall score and the transformed second overall score. The third overall score can be used to make an updated clinical recommendation according to the standardized decision scheme. As described previously, the decision scheme can be based on a cumulative point-value and/or rule-based systems, for example, a first decision based on the third overall score meeting the first criterion associated with the first threshold value, and the like.

In some instances, the decision for intervention, the degree and/or the timing of intervention can depend on non-medical reasons such as health or prior history of the patient, the interest of the patient to undergo the procedure, the level of comfort of the patient to undergo the procedure, financial or personal circumstances of the patient, costs associated with the procedure, a projected trajectory of medical care available, and/or the like.

In some implementations, the transformation can be in the form of generation of a second overall score mapped on to the predetermined scale of the first overall score and associated with the second diagnostic assessment wherein the second diagnostic assessment includes consideration of not only clinical or medical data (e.g., image data) but also other related data such as costs/risk involved in a recommended clinical action or procedure, the state of health of the patient in question, cost considerations for the patient, projected trajectory of health, and or the like. The third diagnostic assessment can be based on integration of the first overall score with the second overall score to generate a third overall score that provides an overall risk assessment. The third overall score can be mapped on the same predetermined scale as the first overall score and the transformed second overall score. The third overall score can be used to make an updated clinical recommendation according to the standardized decision scheme.

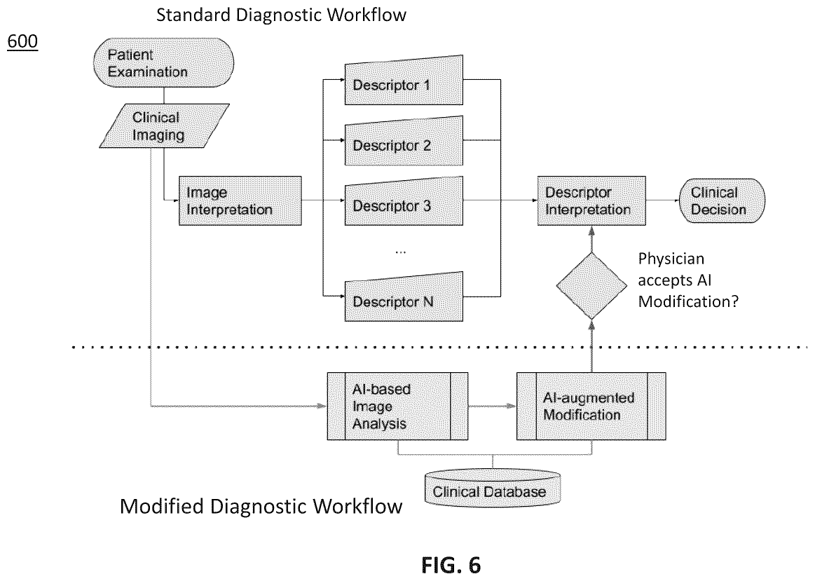

FIG. 6 illustrates an example flowchart of a method 600 describing a method of generating a clinical decision using an AI-based diagnostic assessment and integrating the AI-based diagnostic assessment within a clinical workflow using an AI-based diagnostic system, according to an embodiment. The method 600 can be implemented by a compute device that is similar in structure and/or function to the compute devices 102, 201 and/or analysis devices 105, 305 described above.

As described previously, there are several lexicon-based reporting systems for diagnostic assessments based on standardized decision schemes, such as BI-RADS, TI-RADS, Lung-RADS, etc. which have been proposed and adopted by the medical community. These systems provide a standardized structure for assessing and reporting on suspicious findings discovered in clinical imaging. The flowchart in FIG. 6 below demonstrates the general structure of such a system, and an example method of augmenting such a decision process by integrating an AI-based diagnostic assessment, according to an embodiment.

An I-RADS or other image classification system can apply to many parts of the body (for example, breast, thyroid, prostate, etc.), and many types of clinical imaging (e.g., ultrasound, radiological imaging, etc.), dependent on the body part in question. In assessing a suspicious finding, a physician can determine whether or not an intervention is required, and what is the best type of intervention to be recommended. To reach a decision, the physician can interpret clinical imaging data by considering a list of image descriptors (e.g., descriptor 1, descriptor 2, etc.), each of which can include different options or categories that ca be treated as classes. The features of the image data can be indicative of the tissue or organ of interest in the image data falling under one of the classes with a higher likelihood than the other classes. The higher likelihood of belonging to an identified class can be indicative of a relatively positive or relatively negative prognosis represented by an integer or point value mapped on a predetermined scale (e.g., an integer scale ranging from 0 to 2). These descriptors can be specified by the I-RADS system and can be different for different body parts, anatomies, and/or modalities. For example, while using BI-RADS on a breast lesion imaged by ultrasound, a physician may generate a diagnostic assessment based on a lesion's shape, orientation, echogenicity, margins, and posterior acoustic effects. As another example, while using TI-RADS on ultrasound images of a thyroid nodule, a physician may generate a diagnostic assessment based on a nodule's composition, shape, echogenicity, margins, and echogenic foci.

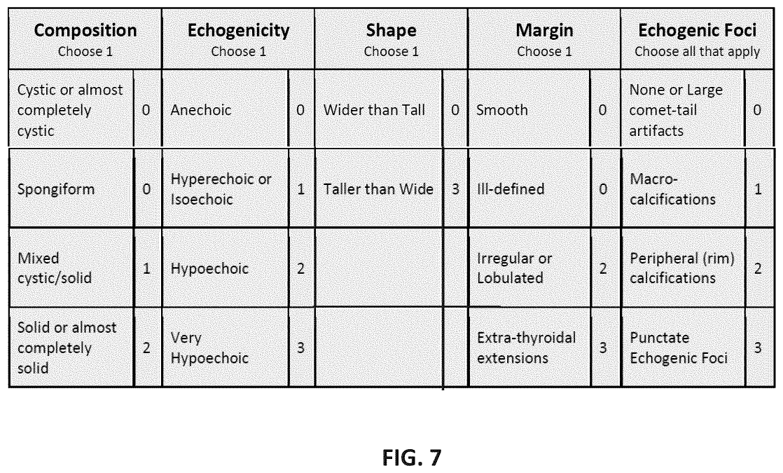

FIG. 7 illustrates an example tabulation of observations that can contribute to a diagnostic assessment based on the TI-RADS system. As an example, the descriptor “Composition” includes four possible classes, namely “Cystic or almost completely cystic”, “Spongiform”, “Mixed cystic/solid”, and “Solid or almost completely solid”. Each class is associated with an integer value or a point value based on the predetermined reporting or classification system used which in this case is TI-RADS. The evaluation of the image data results in the finding of a likelihood, as indicated by the features in the image data of the region (tissue or organ) of interest, that the region (tissue or organ) lies within each of the four listed classes. When the like hood associated with one class is higher than the likelihood associated with the other classes that particular class is selected for that descriptor and the corresponding point value for that selected class is assigned as the point value associated with that descriptor. A similar computation of point values for all the descriptors and a cumulative combination of all the point values for all the descriptors results in an overall score of total points.

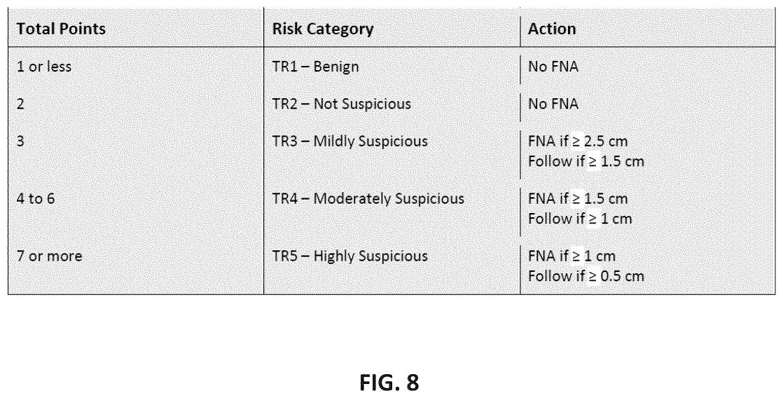

The total points in the overall score can be used to generate a risk assessment based on a rule-based determination of a risk category as shown in FIG. 8. For example, a greater overall score made of total point can indicate a higher risk category which can lead to a recommended clinical action. In some implementations the rule-based determination can be any suitable rule for example a simple threshold rule having a plurality of thresholds such that the overall score crossing each threshold leads to an increment in risk category. Similarly, a threshold value can be used on the estimated risk category to determine recommended clinical action (e.g., No FNA, FNA). As shown, in addition to overall score of total points, in some implementations, additional data or information can used to determine clinical action. The additional data can be any suitable data including size of the cancerous tissue or feature as listed in the table in FIG. 8.

The I-RADS or other image classification system specifies how to interpret the chosen descriptors and determine what type of intervention or clinical action is merited by the finding. Some of these systems are point-based, others are rule-based. An AI-based diagnostic assessment complements a diagnostic assessment using a standardized scheme due to several advantages over the standardized process. First, an AI-based diagnostic assessment can be objective and reproduceable and rule out any subjective bias or error due to subjective determination of results. Second, I-RADS or other image classification system-based assessments are, by definition, limited to the prescribed list of descriptors. AI-based image analysis on the other hand can be used to empirically determine the most significant features (e.g., most statistically significant features in a multi-dimensional feature space) for assessing a potentially suspicious clinical finding. Thirdly, this diagnostic assessment can be achieved with increasing efficacy and/or accuracy via AI-based analysis using machine learning tools implemented using learning algorithms (e.g., supervised, or unsupervised training) on a large database including labeled clinical imaging data tied to pathological ground truth.

As shown in the flowchart in FIG. 6, the I-RADS or other image classification system procedure can be modified via AI-augmented modification to include the AI-based diagnostic assessment alongside the I-RADS or other image classification system defined descriptors. An AI-based interpretation module can determine a suitable method to modify the existing I-RADS or other image classification system decision process with information provided by the AI-based image analysis module. For example, an AI-based interpretation module can be trained on a clinical database with labeled data, tuning a transformation function or a modification function so as to maximize physician performance. Performance can be measured differently depending on modality; an example implementation is described in detail in the following sections.

As a result of this process, the physician is not forced to choose between their own assessment and the AI-system's assessment without access to additional interpretation information. With access to the interpretation module, physicians can be able to leverage both assessments to optimal effect. While described here an I-RADS or other image classification system-based image interpretation performed by a physician which is modified to integrate an AI-based diagnostic assessment, the method described herein is just as applicable if performed by another AI system in place of the physician, and configured to integrate two dissimilar AI-based diagnostic assessments.

### Example: Implementation of an AI-Based Diagnostic System to Analysis of Thyroid Ultrasound Data

Thyroid nodules occur in up to 68% of individuals. Most nodules (˜95%) are benign and many malignant nodules may not result in symptoms or death. Still, over 600,000 FNAs are performed annually in the U.S. ACR TI-RADS was developed to standardize diagnostic criteria, reduce biopsy rates, and limit the overdiagnosis of thyroid cancer. TI-RADS increases reader (e.g., radiologist, physician, etc.) concordance while reducing unnecessary biopsies by 19.9-46.5%.

### ACR TI-RADS

ACR TI-RADS provides a standardized consistent framework with which to determine the risk posed by a thyroid nodule, and whether to perform a FNA or a follow up examination. In some instances, the first step in this framework is to characterize the nodule in question across the five descriptor categories shown in the table in FIG. 7.

As indicated in the table in FIG. 7, each choice carries a point value. The TI-RADS point total obtained by summation of these points is correlated to the nodule's likelihood of malignancy and is used to determine the nodule's risk category. The calculation based on the table, along with the nodule's size is used to determine the course of action for treatment, as described by the rules in the table in FIG. 8.

While ACR TI-RADS is an effective system, it has a few limitations. The first limitation is that the judgments made on the composition, echogenicity, shape, margin, and echogenic foci categories are all subjective determinations made by the interpreting physician. Small differences in opinion, such as anechoic versus very hypoechoic (two conditions that can appear quite similar in an ultrasound image) produce a large change in outcome. The second limitation is that the approach is limited in scope to the five specified feature categories. The rigidity of ACR TI-RADS precludes consideration of any imaging features beyond these specific categories.

### AI-Based Diagnostic System

An AI-based diagnostic system according to the disclosed embodiments (e.g., system 100) has the advantage of approaching the problem with no preconceptions about relevant imaging features, where optimal features are learned directly from the data by the system and considered holistically when evaluating a nodule. The AI-based diagnostic system is therefore capable of rendering an estimate of likelihood of malignancy of a region of interest with much higher accuracy than physicians using the ACR TI-RADS system alone.

Advances in AI-based techniques have increased opportunities to further improve the ACR TI-RADS system. In this study, the use of an additional AI-generated nodule risk descriptor was analyzed to independently assess risk of malignancy. Such an analysis and independent assessment can be carried out by an AI-based diagnostic system and/or components within an AI-based diagnostic system (e.g., system 100, compute device(s) 102, 201, physician device 103, and/or analysis device 105, 305) described herein. For example, all or portions of the analyses can be carried out by a compute device (e.g., compute device 102, 201) and/or an analysis device (e.g., analysis devices 105, 305) described herein. A predictive indicator generated using an AI-based diagnostic system was subsequently mapped to an integer point value ranging from −2 to +2 to be incorporated into the already-established TI-RADS point-based clinical management criteria to improve patient management decisions. The AI-based diagnostic system was configured to prepopulate TI-RADS descriptors generating a putative point total that a reader can then consider and modify at their discretion.

Using ACR TI-RADS, physicians have extremely clear guidelines on how to evaluate a thyroid nodule, and no consistent mechanism for considering external information. Indeed, early research indicates that if presented with an AI-based diagnosis that conflicts with an ACR TI-RADS diagnosis, a physician is most likely to disregard the AI diagnosis, and stick to the ACR TI-RADS guidelines even when the physician accepts that the AI system has higher potential performance. An AI-based diagnostic system as described herein provides a solution to this problem by transforming the AI-based diagnostic assessment into a new feature category, included in a modified diagnostic assessment, to be considered among the ACR TI-RADS's existing five categories, allowing the system to provide a direct update to the physician's ACR TI-RADS point total. This can be graphically summarized as shown in in FIGS. 9A and 9B and described below.

FIG. 9B shows an example interface displaying a first diagnostic assessment (e.g., a physician-based diagnostic assessment) and FIG. 9A shows an example interface displaying a third diagnostic assessment following an integration of the first diagnostic assessment of FIG. 9B and a transformed second diagnostic assessment (e.g., an AI-based diagnostic assessment, not shown). The first diagnostic assessment and the third diagnostic assessment shown in FIGS. 9B and 9A are based on the established or standardized classification/reporting system TI-RADS. Each of the first and third diagnostic assessments in FIGS. 9B and 9A, respectively, includes an overall score (901B and 901A, respectively) and a set of descriptors 903 with each descriptor in the first diagnostic assessment and the third diagnostic assessment being associated with a point value (e.g., 904B, 904A, respectively) The TI-RADS overall score 901B is based on physician based analysis and the overall score 901A is based on AI-based analysis, each computed based on the associated descriptors and mapped on a predetermined scale of 1 to 5 (or TR1 to TR5) each level indicating a risk category, as described herein. The overall score and the risk category are displayed via a user interface (e.g., a user interface coupled to or integrated in a compute device 102, 201 and/or analysis device 105, 305). The user interface includes information related to the diagnostic assessment computed and a clinical recommendation based on the diagnostic assessment.

In the example interface illustrated in FIGS. 9A and 9B, expanded representation of the output for each descriptor is shown when clicking on a descriptor category. The descriptor selected by the system is highlighted in blue. In some embodiments, the user interface can be configured to provide a control tool 908 (e.g., a clickable selection device that opens a collapsible drop-down list) that can be activated to reveal information associated with the third diagnostic assessment. The control tool 908 can be clicked again to collapse the information. For example, as shown in FIG. 9A, an activation of the control tool can open a portion of the user interface configured to provide information associated with the transformed second diagnostic assessment (AI-based diagnostic assessment).

The transformed second diagnostic assessment is in the form of a TI-RADS point modifier, generated by mapping probability ranges from the AI-based diagnostic system used for Thyroid data analysis to integer point modifiers ranging from −2 to +2. If the transformed second diagnostic assessment 910 transitions an initial TI-RADS score to a new TI-RADS category, the original and new category are visually displayed on the CLI bar 905. The point thresholds for TI-RADS scores (e.g., thresholds indicated by vertical gray lines) are also shown in the colored bar 905. Optionally, readers (e.g., physicians) can choose to disable the modifier by clicking on a control tool 909 and collapsing it, making it gray.

The transformation is designed to optimally impact physician decision making. Specifically, it is designed to affect a maximal positive impact to the physician's Area Under the ROC Curve (AUC), sensitivity, and specificity. This approach has two key advantages over existing AI systems. First, it is easily incorporated into the ACR TI-RADS system. Second, rather than forcing a physician to choose between their own judgment and the system's overall recommendation, it provides an optimal combination of the two, which is potentially stronger than either system is individually.

As shown in FIG. 9A, selection of one of the descriptors (e.g., SHAPE) can display a set of classes (options or categories) 907A under that descriptor (e.g., Wider-Than-Tall, Taller-Than-Wide) and display a graphical representation of a probability or likelihood associated with each class. For example, the user interface can display, for each class of a descriptor, a bar 907A filled with a color, the extent of filling corresponding to the likelihood. In some implementations, the processor implementing the user interface can evaluate the likelihood or probability associated with each class against a predetermined probability (e.g., a threshold probability or a random chance probability given the number of classes for that descriptor) and generate the CLI based on the evaluation. The class with highest likelihood and/or maximal likelihood greater than a specified threshold is highlighted in a blue color.

In some embodiments, the user interface displaying a third diagnostic assessment, which is an integration of a physician based first diagnostic assessment and a transformed AI-based second diagnostic assessment, can be configured to provide a control tool 909 (e.g., a clickable selection device) that opens a collapsible dropdown display 910 that can be activated to reveal information associated with the transformed second diagnostic assessment that was used to generate the third diagnostic assessment by updating the first diagnostic assessment. The control tool 909 can be clicked again to collapse the information in the portion 910 (collapses state as shown in FIG. 9B). For example, as shown in FIG. 9B, the selection of a descriptor (e.g., SHAPE) and the activation of the control tool can open the bottom portion of the user interface to display a graphical representation of the predetermined scale used to map the point-values of each descriptor and display, in a vector or arrow form, the first point value and the second point value associated with that descriptor based on the first and second diagnostic assessments respectively, and the difference between the two. In some implementations, the graphical representation of the predetermined scale used to map the point-values can include a color map 905A with, for example, the colors ranging from green to red and hotter colors corresponding to a greater severity of diagnosis or greater risk category.

In some embodiments, the user interface can display as part of the third diagnostic assessment, a confidence level indicator (CLI) associated with the adjustment or modification provided for each descriptor and/or the overall score. In some implementations, the CLI can be computed by a model trained to generate the CLI associated with an image data with reference to a specified classification or reporting system (e.g., TI-RADS, BI-RADS, etc.) In some instances, the model can be similar in structure and/or function not the ML model 355. The model can be trained to process an image (e.g., an image containing a lesion) by extracting features from the image in a wholistic manner (e.g., in a manner not constrained by any definition of descriptors) and comparing the features to those of labeled reference images from repository or library, the labeling being based on the categories defined by the specified classification or reporting system. Example methods of determining a CLI are described in U.S. Pat. No. 9,536,054, incorporated above by reference. For example, the labeled reference images can be labeled to belong to one of the several categories of severity of a diagnosis ranging from TR1 to TR5 according to the TI-RADS system. Based on the output of the model, the image can be assigned a CLI that can be graphically represented in the user interface as shown by the CLI 906A indicated by the inverted white arrowhead placed along the blue line.

As shown in FIG. 9A, the first point value associated with the descriptor “SHAPE” based on the physician based first diagnostic assessment was 0 indicated by the starting point of the blue arrow 906A. The second point value associated with the descriptor “SHAPE” based on the transformed AI-based second diagnostic assessment was −1 indicated by the end point of the blue line. The CLI 906A depicted by the inverted white arrow provides the confidence level associated with the adjustment to −1 based on the transformed AI-based second diagnostic assessment. The location of the CLI 906A, by virtue of being closer to the category TR1 (TI-RADS 1) associated with an adjustment of −2 indicated by the green (leftmost) bar on the color bar 905A, than to the category TR3 (TI-RADS 3) associated with an adjustment of 0 indicated by the yellow bar on the color bar 905A (i.e., to the left of a midpoint along the light green bar associated with the category TR2 with score −1), shows a higher confidence level (e.g., likelihood) of the region of interest being associated with TR3 (0) than with TR2 (−1). The difference between the first point value and the second point value associated with that descriptor is also provided adjacent to the control tool 909.

In some embodiments, the overall score diagnostic assessment is presented in a point-based system with greater numbers indicating a higher risk category with a higher degree of severity of the diagnosis. For example, as shown in FIGS. 9A and 9B, an overall score of 2 can be associated with a finding or risk category “Not Suspicious” whereas an overall score of 3 can be associated with a finding or risk category of “Mildly Suspicious.” In some embodiments, the degree of severity and/or the risk category of the diagnosis can also be indicated by a color code in a specified color scheme, as shown by the green and yellow bands at the top portions of FIGS. 9A and 9B, respectively. For example, the indication of degree of severity/risk category can be associated with a color chosen from a specified color scheme ranging from green to red with hotter/redder colors corresponding with increased degrees of severity. Thus, an overall score of two can be associated with a green band as in FIG. 9A, and an overall score of 3 can be associated with the yellow band as shown in FIG. 9B. The color scheme associated with the representation of the overall score can be the same as the color map 905A associated with the graphical representation of the predetermined scale used to map the point-values associated with each descriptor described above.

In some implementations, as in the example shown in FIGS. 9B and 9A, the clinician or physician based first diagnostic assessment can provide a first overall score 901B which can be different from the overall score 901A provided by the third diagnostic assessment. by a highlighted blue and white indicator 906A as shown in FIG. 9A.

The user interface can display a clinical recommendation based on the diagnostic assessment. The clinical recommendation can include one of a biopsy or no biopsy or any suitable follow up procedure or clinical assessment as needed. Some example clinical recommendations can include a fine needle aspiration (FNA) which can be one type of biopsy. As shown in the examples in FIGS. 9A and 9B, the clinical recommendation can be based on the diagnostic assessment and the resulting degree of severity. A point value of two indicating a not suspicious tissue image can result in a clinical recommendation of “No FNA” as in FIG. 9A. A point value of 3 indicating a mildly suspicious tissue image (of potential cancerous nature) can result in a clinical recommendation of an FNA. The clinical recommendation of “No FNA”, “Follow-Up”, or “FNA” can be displayed alongside the point total with a breakdown of the points based on descriptors included below. In some embodiments, the user interface can also include the list of descriptors and, in the top portion, information associated with one or more descriptors such as 902A and 902B (for example when that descriptor, shape/size, is selected). In some implementations, the user interface can be configured to receive input from a user to update one or more point values and/or descriptors associated with a diagnostic assessment that is being displayed. For example, a user (e.g., a reader, physician, clinician, etc.) can modify or update pre-populated descriptors, classes, and/or point values associated with the descriptors based on their clinical judgement.

If the transformed second diagnostic assessment shown in portion 910 (integrating the AI-based diagnostic assessment transformed to map to the physician based diagnostic assessment) is included, the modified overall score can be shown with the adjustment (e.g., “2 pts=3 pts−1”). TI-RADS Size Criteria 902A and 902B indicate the nodule size (shown in bold), compared to size criteria, if applicable, for clinical recommendation based on ACR TI-RADS guidelines. The Confidence Level Indicator (CLI) 906A can be per descriptor 907A, in some instances, indicating the confidence level associated with that descriptor and//or a class of that descriptor. In some embodiments, the CLI 906A provided per descriptor can be determined using a machine learning model or AI-based model trained to generate a confidence level specific to a particular descriptor using labeled reference images and/or including previous clinical assessments, CAD-based or AI-based assessments, and/or ground truth data (e.g., biopsy data). In some instances, a CLI 906A can be provided for a total point valve associated with the cumulative point value based on all descriptors instead of each individual descriptor. In some instances, the Confidence Level Indicator (CLI) 906A can be provided for a particular region of interest (e.g., based on its image data) belonging to a particular classification (e.g., TI-RADS 1, TI-RADS 2, etc.). Example methods of determining a CLI are described in U.S. Pat. No. 9,536,054, incorporated above by reference.

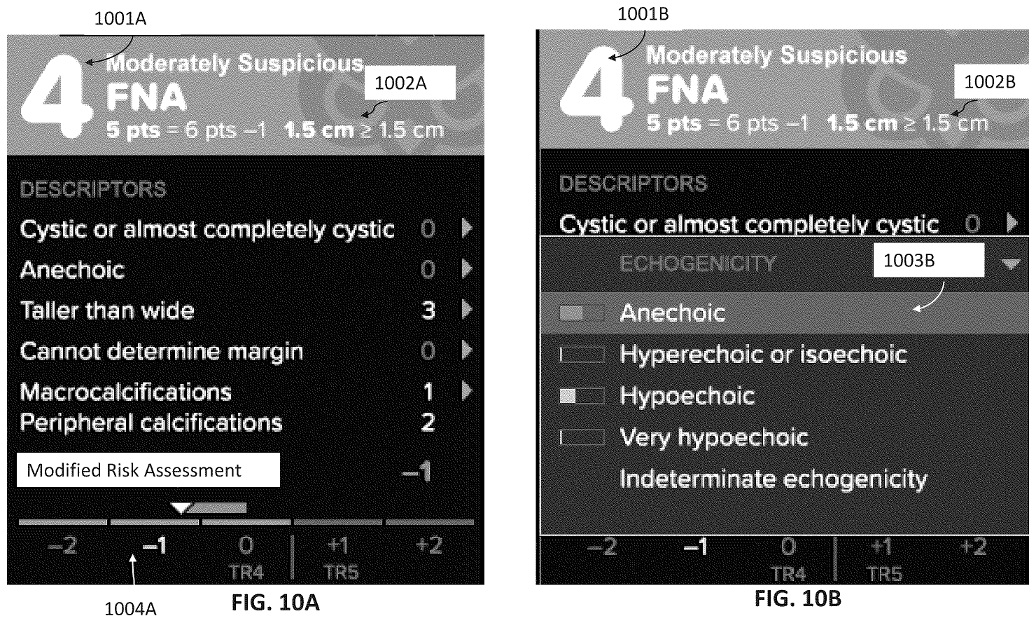

FIGS. 10A and 10B are examples of a user interface displaying diagnostic assessments and clinical recommendations, as described herein. The user interface of FIGS. 10A and 10B can be structurally and/or functionally similar to the user interface in FIGS. 9A and 9B and therefore include similar features. The degree of severity indicated in FIGS. 10A and 10B is associated with an overall score of four 1001A and 1001B and a finding of “Moderately Suspicious” as indicated by the hotter or redder color of band, along the color scheme, displayed in the top portion of the interface. The color scheme used can be modified based on the degree of severity and the color bar used to indicate the range can be updated accordingly. For example, the color scheme used in FIGS. 11A and 11B shows smaller range of colors (orange to red) representing the point value system. As described previously, each descriptor can be selected and updated or edited by a user to result in an updated diagnostic assessment. The selection of a descriptor (e.g., echogenicity) reveals a list of classes that are associated with the descriptor as shown by the list 1003B, and a graphical representation of a probability associated with each class, and the class with highest probability highlighted in blue, as described previously with reference to FIG. 9A. Assignment of a particular value to a descriptor results in updating the point value associated with that descriptor which in turn results in updating the overall score 1001A and 1001B associated with that particular diagnostic assessment, respectively.

In some implementations, the transformation of the AI-based second diagnostic assessment can be in the form that provides only a second overall score (rather than a second set of points values associated with the descriptors). The second overall score can be integrated with the first overall score associated with the physician based first diagnostic assessment to generate a third overall score indicating a degree of severity of a diagnosis or a risk category associated with the diagnosis. The third overall score can be used to recommend clinical action based on a predetermined decision scheme.

In some implementations, the transformation of the AI-based second diagnostic assessment can be in the form that provides a second overall score that is based not on medical image data but also on other non-image data as well as potentially from non-medical or non-clinical data (e.g., biographic information, patient history, cost considerations, availability of care, projected trajectory of progression of health, etc.). The second overall score can be integrated with the first overall score associated with the physician based first diagnostic assessment to generate a third overall score indicating an overall risk assessment and risk category associated with the diagnosis, as shown in FIG. 10A. The third overall score indicating the overall risk assessment (e.g., shown in FIG. 10A) can be used to recommend clinical action based on a predetermined decision scheme.

In an example method of incorporating an AI-based diagnostic assessment into the decision-making process can be accomplished using an AI-based diagnostic system (e.g., system 100 and/or components thereof) as follows:

1. Obtain a dataset of ultrasound images of thyroid nodules. The dataset can include
a. one or more images of each nodule (two orthogonal views is typical)
b. ground truth labels for each nodule (benign/malignant)
c. size measurements for each nodule (this criterion is specific to thyroid).

2. Have one or more qualified readers provide TI-RADS assessments, following the ACR TI-RADS guidelines, for the nodules in the dataset. Several assessments from several readers can provide an improved aggregate, since these reader-based assessments are subjective and evaluating several readers can mitigate the effects of inter-reader variability. Readers can include both trained physicians and AI systems trained to produce TI-RADS assessments. For TI-RADS, these assessments can include:
a. Composition
b. Echogenicity
c. Shape
d. Margin
e. Echogenic Foci
f. Total TI-RADS points (determined from a-e)
g. TI-RADS Risk Category (determined from f)
h. Decision to perform a biopsy or FNA (determined by g and nodule size)

3. Calculate the desired performance metrics for each of the readers. In some implementations, an analysis device (e.g., analysis device 105, 305) can perform an evaluation (e.g., via assessment evaluator 359) to calculate performance metrics associated with assessments of each of the readers. Desired performance metrics can include the aspects of performance that are desired to be maximized. In one implementation, the desired performance metrics can include AUC, sensitivity, and specificity.

A binary prediction can be made using a preselected threshold value and based on measuring a threshold crossing data point or event (for example, an identification of a data point as positive if the data point crosses a threshold value for a specified feature and identifying the data point as negative if the data point does not cross the threshold value). When a binary prediction is made there can be four types of outcomes: (1) True Negative (TN): correct prediction that the class is negative, (2) False Negative (FN): incorrect prediction that the class is negative, (3) False Positive (FP): incorrect prediction that the class is positive, and (4) True Positive (TP): correct prediction that the class is positive.

| | | Predicted Class | |
|---|---|---|---|
| | | Class 0 (negative) | Class 1 (positive) |
| Actual | Class 0 (negative) | TN | FP (Type-I Error) |
| Class | Class 1 (positive) | FN (Type-II Error) | TP |

A first metric, True positive rate (TPR), also referred to as sensitivity, can be defined as TP/(TP+FN). The TPR metric can correspond to the proportion of positive data points that are correctly considered as positive, with respect to all positive data points. A second metric, False positive rate (FPR), also referred to as (1−specificity), can be defined as FP/(FP+TN). The FPR metric can correspond to the proportion of negative data points that are mistakenly considered as positive, with respect to all negative data points.

In some implementations, the FPR and the TPR can be combined into one single metric, by computing the two former metrics (FPR and TPR) with many different thresholds (for example 0.00, 0.01, 0.02, . . . , 1.00) for the logistic regression, then plotting them on a single graph, with the FPR values on the abscissa and the TPR values on the ordinate. The resulting curve is called ROC curve. The ROC curve can be analyzed and/or quantified using a metric called the Area Under the Curve of this curve, also referred to as the AUC-ROC.

The AUC-ROC (Area Under the Curve-Receiver Operating Characteristic curve) is a graphical representation of sensitivity, as represented by rate or frequency of true positive findings, versus a measure of specificity (1−specificity), represented by a rate or frequency of false positive findings. As described above, ROC curves are graphs in which the true positive rate (TPR) is plotted on the Y axis and the false positive rate (FPR) is plotted on the X axis. Graphs with ROC curves can represent the relative tradeoffs between benefits (true positives, sensitivity) and costs (false positives, 1−specificity). An increase in sensitivity may be accompanied by or at the cost of a decrease in specificity.

4. Train a machine learning model in the AI-based diagnostic system to independently assess a risk of malignancy of nodules based directly on the image data. This AI-based system takes as input all of the image data tied to a specific nodule, and produces a risk estimate correlated to the likelihood of malignancy for that nodule. In one implementation, that estimate can take the form of a floating-point number between 0 and 1.

5. Generate a parameterized transformation function, fₜᵣₐₙₛ, with parameters θ, which maps the system's output to a TI-RADS point value. Many such functions can be designed and used. In the example function that is shown below, x is the system's raw output, θ are the function's parameters, and a are the allowed output values for the transformation.

transformed output=fₜᵣₐₙₛ(x,θ,α)

In one implementation, ∈{−2,−1,0,+1,+2}

6. Define an objective function which can be used to optimize fₜᵣₐₙₛ(x,θ,α). This function can take a variety of forms, so long as it relates the change in reader performance to the parameters of the transformation function. In one implementation, as described in previously, several clinically relevant metrics were used to characterize performance and create an objective. In another implementation, an algorithm learns an optimal objective from the data. The objective function can be designed to suit the specific requirements of the diagnostic system being modified. The objective function used in this implementation is defined as

$$Objective = \frac{1}{♯ of readers} {\sum }_{readers} {λ}_{1} d ( {AUC}_{updated} ( θ ) , {AUC}_{target} ) + {λ}_{2} d ( {PPV}_{updated} ( θ ) , {PPV}_{target} ) + {λ}_{3} d ( {NPV}_{updated} ( θ ) , {NPV}_{target} ) + {λ}_{4} d ( {Sens}_{updated} ( θ ) , {Sens}_{target} ) + {λ}_{5} d ( {Spec}_{updated} ( θ ) , {Spec}_{target} ) + {λ}_{6} d ( {PLR}_{updated} ( θ ) , {PLR}_{target} ) + {λ}_{7} d ( {NLR}_{updated} ( θ ) , {NLR}_{target} )$$

where d(a,b) is a distance function.

The value of each A term can be chosen to reflect the relative importance of each performance metric and account for the average magnitude of each of the performance metrics used. The above-described objective function can be used to maximize the transformation function so that it is able to provide the maximal positive impact in terms of AUC, sensitivity, and specificity.

7. Compute modified diagnostic assessment or a modified TI-RADS point totals for each of the readers, using the transformed system output, as a function of the parameters, θ, of the transformation function.

points_{updated}=points_{tirads:}(x,θ)+f_{trans:}(x,θ,α)

In another implementation, a multiplicative transformation can be applied as follows.

points_{updated}=points_{tirads}(x,θ)*fₜᵣₐₙₛ(x,θ,α)

More generally,

points_{updated}=m(points_{tirads}(x,θ),fₜᵣₐₙₛ(x,θ,α))
Where m(a,b) is a modification function appropriate to the diagnostic system in question.

8. From the updated point total, calculate updated performance metrics, and compute the value of the objective function used. This process can be performed over multiple datasets, for TIRADS point totals coming from multiple readers. A multitude of readers and datasets can be used to improve the generalizability of the results.

9. Optimization techniques are used to determine the optimal values of the function parameters, θ, which maximize the objective function. Many optimizers can be used for this process such as genetic algorithms, NOMAD, SGD. Our specific implementation involves Bayesian Optimization, also known as Gaussian Process Regression.

Once the optimization process is finished, the resulting transformation function provides a point modification on a scale appropriate to the system being augmented that optimally impacts the behavior of an individual making a clinical assessment. In one implementation for ACR TI-RADS, the scale was −2 to +2 and the transformation function improves the individual's AUC, sensitivity, and specificity, and by extension reduces the number of benign biopsies performed. Typically, this individual can be a physician. The approach, however, is equally valid for any entity performing a diagnosis, including other AI devices.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

FIGS. 11A and 11B illustrate the improvement in diagnostic performance provided by the AI-based diagnostic system as described herein, in a multi-reader, multi-case study involving 15-readers (11-radiologists, 4-endocrinologists) and 650 FNA-proven nodules (130 malignant). Readers evaluated each nodule twice across two sessions separated by a 4-week washout period. In each session, nodules were randomly presented and evaluated in one of two conditions:
1) Manual scoring of a TI-RADS report form (TI-RADS Only)
2) AI-prepopulated TI-RADS report form, augmented with an AI-based risk descriptor and point modifier (TI-RADS+AI).

Assuming a recommendation for FNA was a positive result, diagnostic performances in the two reading conditions were assessed via parametric AUC-ROC and operating point analyses. Operating point analyses included a comparison of sensitivity, specificity, positive predictive value (PPV), and negative predictive value (NPV) between reading conditions, related to the physician's recommendation to biopsy. Inter-reader variability was assessed via Pearson's R correlation. Inter-reader variability can be important as in practice, two different physicians may give different assessments of the same finding. These assessments can be subjective. Reducing inter reader variability can thus mean more consistent diagnoses for patients. Interpretation time was analyzed as a relative change between reading conditions. Interpretation time is important because it impacts how many cases a physician is able to effectively analyze in a working day.

As shown in FIG. 1A, average AUC improvement for TI-RADS+AI (i.e., TI-RADS, as used by a physician, plus a model such that those used in the methods described herein) versus TI-RADS was 0.083 (95% CI, 0.066-0.099). FIG. 11A shows per reader parametric AUC comparing TI-RADS Only to TI-RADS+AI for all readers on all of the data. The dashed line represents equivocal results with all points above this line demonstrating an improvement for the TI-RADS+AI reading condition. Table 1

Table 1 shows data associated with each reader and average performance with 95% confidence intervals for the parametric analysis.

| | | |
|---|---|---|
| Reader | Difference in AUC | Percent Change in AUC |

| | | | | |
|---|---|---|---|---|
| R1 | 0.076 | [0.011, 0.141] | 10.442 | [1.541, 19.344] |
| R2 | 0.047 | [−0.021, 0.114] | 6.072 | [−2.748, 14.893] |
| R3 | 0.100 | [0.033, 0.167] | 13.765 | [4.446, 23.085] |
| R4 | 0.054 | [−0.016, 0.125] | 7.326 | [−2.212, 16.864] |
| R5 | 0.087 | [0.026, 0.147] | 11.871 | [3.541, 20.201] |
| R6 | 0.114 | [0.052, 0.176] | 15.735 | [7.053, 24.416] |
| R7 | 0.051 | [−0.014, 0.115] | 6.593 | [−1.765, 14.951] |
| R8 | 0.105 | [0.039, 0.171] | 14.742 | [5.420, 24.063] |
| R9 | 0.059 | [−0.004, 0.122] | 7.913 | [−0.514, 16.340] |
| R10 | 0.139 | [0.072, 0.205] | 20.685 | [10.641, 30.729] |
| R11 | 0.079 | [0.016, 0.142] | 10.593 | [2.068, 19.118] |
| R12 | 0.073 | [0.008, 0.138] | 9.781 | [1.057, 18.505] |
| R13 | 0.088 | [0.025, 0.152] | 12.079 | [3.327, 20.831] |
| R14 | 0.109 | [0.049, 0.168] | 15.128 | [6.753, 23.504] |
| R15 | 0.065 | [−0.003, 0.133] | 8.892 | [−0.387, 18.170] |
| Average | 0.083 | [0.066, 0.099] | 11.386 | [9.119, 13.652] |

Change in sensitivity and specificity of follow-up recommendations for all data for all readers is shown in FIG. 11B. The symbol for each subject (R1, R2, R3, etc.) at the base of the arrow represents the initial operating point, while the same symbol at the arrowhead represents the operating point of using the TI+RADS+AI, with arrows pointing to the top right indicating an increase in sensitivity and specificity as a result of using TI-RADS+AI. As shown in FIG. 10B, TI-RADS+AI also produced a significant increase in sensitivity and specificity of 8.4% (95% CI, 5.4%-11.3%) and 14% (95% CI, 12.5%-15.5%), respectively. Inter-reader variability was 0.622 and 0.876 for TI-RADS and TI-RADS+AI, respectively. Interpretation time decreased by 23.6% (p<0.001) for TI-RADS+AI.

Table 2 shows the change in sensitivity and specificity that is graphically depicted in FIG. 11B.

TABLE 2

| Reader | Difference in Sensitivity | | Percent Change in Sensitivity | |
|---|---|---|---|---|
| R1 | 0.056 | [−0.160, 0.273] | 8.795 | [−24.964, 42.554] |
| R2 | 0.140 | [−0.053, 0.332] | 22.748 | [−9.104, 54.600] |
| R3 | 0.235 | [0.037, 0.433] | 45.702 | [5.155, 86.249] |
| R4 | −0.048 | [−0.257, 0.161] | −6.684 | [−35.733, 22.365] |
| R5 | 0.076 | [−0.102, 0.254] | 12.093 | [−16.277, 40.462] |
| R6 | −0.023 | [−0.209, 0.163] | −2.874 | [−25.819, 20.071] |
| R7 | 0.053 | [−0.135, 0.240] | 7.449 | [−19.138, 34.037] |
| R8 | 0.147 | [−0.033, 0.327] | 21.617 | [−5.235, 48.469] |
| R9 | −0.052 | [−0.237, 0.133] | −7.090 | [−32.441, 18.261] |
| R10 | 0.130 | [−0.053, 0.313] | 24.453 | [−10.371, 59.278] |
| R11 | 0.246 | [0.054, 0.439] | 47.215 | [8.359, 86.071] |
| R12 | 0.081 | [−0.116, 0.278] | 12.469 | [−18.045, 42.983] |
| R13 | 0.236 | [0.057, 0.416] | 40.972 | [8.490, 73.454] |
| R14 | 0.048 | [−0.140, 0.236] | 7.500 | [−21.697, 36.696] |
| R15 | 0.058 | [−0.127, 0.242] | 8.340 | [−18.273, 34.954] |
| Average | 0.092 | [0.043, 0.141] | 16.180 | [8.204, 24.156]. |

| Reader | Difference in Specificity | Percent Change in Specificity | |
|---|---|---|---|
| R1 | −0.037 [−0.137, 0.063] | −6.779 | [−24.943, 11.385] |
| R2 | 0.122 [0.042, 0.203] | 20.079 | [6.670, 33.488] |
| R3 | 0.328 [0.252, 0.405] | 65.691 | [48.127, 83.256] |
| R4 | 0.231 [0.145, 0.318] | 44.408 | [26.949, 61.867] |
| R5 | 0.246 [0.162, 0.330] | 47.446 | [30.197, 64.695] |
| R6 | 0.317 [0.224, 0.410] | 69.661 | [46.151, 93.171] |
| R7 | 0.231 [0.148, 0.313] | 39.384 | [24.610, 54.158] |
| R8 | 0.386 [0.302, 0.469] | 90.398 | [65.633, 115.164] |
| R9 | 0.182 [0.091, 0.274] | 34.249 | [16.447, 52.051] |
| R10 | 0.190 [0.116, 0.264] | 29.425 | [17.586, 41.263] |
| R11 | 0.169 [0.095, 0.243] | 26.015 | [14.313, 37.716] |
| R12 | 0.280 [0.203, 0.358] | 53.200 | [37.135, 69.264] |
| R13 | 0.328 [0.256, 0.399] | 66.830 | [50.256, 83.404] |
| R14 | 0.369 [0.282, 0.457] | 83.511 | [59.417, 107.606] |
| R15 | 0.289 [0.198, 0.379] | 63.595 | [41.091, 86.100] |
| Average | 0.242 [0.220, 0.264] | 48.474 | [43.751, 53.197] |

In conclusion, automated AI-based pre-population of the 5 TI-RADS descriptors combined with use of an additional AI-based risk descriptor and point modifier to generate a modified diagnostic assessment significantly improved reader diagnostic accuracy while simultaneously decreasing interpretation time and inter-reader variability.

Other embodiments may include augmenting the American Thyroid Association (ATA) guidelines for assessment of thyroid nodules through analysis of the sonographic pattern of the nodule for risk stratification or the ACR BI-RADS lesion categorizations and risk stratifications using a modified diagnostic assessment on an AI-based diagnostic system (e.g., system 100) described herein.

Although various embodiments have been described as having particular features and/or combinations of components, other embodiments are possible having a combination of any features and/or components from any of embodiments as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In this disclosure, references to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. The use of any and all examples, or exemplary language (“e.g.,” “such as,” “including,” or the like) provided herein, is intended merely to better illuminate the embodiments, and does not pose a limitation on the scope of the embodiments or the claims.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A method, comprising:
receiving, at a compute device, image data associated with a region of interest;
receiving, at the compute device, a first diagnostic assessment associated with the image data;
receiving, at the compute device, a second diagnostic assessment associated with the image data, the second diagnostic assessment being different from the first diagnostic assessment; and
integrating the second diagnostic assessment with the first diagnostic assessment to generate a third diagnostic assessment associated with the clinical data.

2. The method of claim 1, further comprising:
presenting, via an interface of the compute device, at least one of the first diagnostic assessment or the third diagnostic assessment.

3. The method of claim 1, wherein the first diagnostic assessment is provided by a user based on analysis of the image data by the user, and the second diagnostic assessment is generated by processing the image data using a machine level model trained to classify the region of interest based on one or more image features.

4. The method of claim 3, wherein the machine learning model includes one or more of: a deep neural network, a multi-layer perceptron, a random forest, a support vector machine.

5. The method of claim 1, wherein the first diagnostic assessment is in a first format and the second diagnostic assessment is in a second format, the integrating including:
applying a transformation function to the second diagnostic assessment to generate a transformed second diagnostic assessment prior to integrating the transformed second diagnostic assessment with the first diagnostic assessment to generate the third diagnostic assessment.

6. The method of claim 1, wherein at least one of the first diagnostic assessment or the second diagnostic assessment is generated according to a predefined image classification system.

7. The method of claim 6, wherein the predefined image classification system includes at least one of a standardized system under a class of Reporting and Data Systems (RADS) organized by the American College of Radiology or a standardized system organized by the American Thyroid Association.

8. The method of claim 1, wherein the region of interest includes a lesion, and at least one of the first diagnostic assessment or the second first diagnostic assessment includes an indication of a degree of malignancy of the lesion determined based on an image classification system.

9. The method of claim 1, wherein the first diagnostic assessment is a based on a first set of values associated with one or more descriptors related to the region of interest, and the transformed second diagnostic assessment is based on a second set of values associated with the one or more descriptors related to the region of interest, the method further comprising:
generating a confidence level indicator associated with each value of the second set of values for each descriptor from the one or more descriptors, the confidence level indicator configured to indicate a level of confidence associated with the value associated with each descriptor in generating the transformed second diagnostic assessment.

10. The method of claim 1, wherein the first diagnostic assessment is associated with a first score under a predefined classification system, the method further comprising:
presenting, via an interface of the compute device, (1) the third diagnostic assessment associated with a second score under the predefined classification system and (2) a difference between the first and second scores.

11. An apparatus, comprising:
a memory; and
a processor operatively coupled to the memory, the processor configured to:
receive image data associated with a region of interest;
receive a first diagnostic assessment associated with the image data, the first diagnostic assessment in a first format and based on a set of first values assigned to one or more descriptors associated with the image data;
process the image data using a machine learning (ML) model to generate an output indicating a second diagnostic assessment associated with the clinical data, the second diagnostic assessment in a second format;
transform the second diagnostic assessment from the second format to the first format; and
generate a third diagnostic assessment by integrating the transformed second diagnostic assessment with the first diagnostic assessment, the transformed second diagnostic assessment being integrated in the form of a set of second values assigned to each descriptor from the one or more descriptors based on the second diagnostic assessment.

12. The apparatus of claim 11, wherein the processor is further configured to:
present, via a display, a user interface configured to present clinical recommendations based on diagnostic assessments;
display, via the user interface, the first diagnostic assessment, the first diagnostic assessment including at least one of a points-indication of a first degree of severity associated with the region of interest or a first clinical recommendation based on the first degree of severity associated with the region of interest; and
display, via the user interface, an indication of an availability of the third diagnostic assessment, and a control tool configured to be activated by a user to show information associated with the third diagnostic assessment.

13. The apparatus of claim 12, wherein the processor is further configured to:
receive a signal indicating an activation of the control tool requesting the information associated with the third diagnostic assessment; and
display, via the user interface and in response to the signal, the third diagnostic assessment, the third diagnostic assessment including at least one of a points-based indication of a second degree of severity associated with the region of interest and a second clinical recommendation based on the second degree of severity associated with the region of interest.

14. The apparatus of claim 11, wherein the one or more descriptors are based on a predefined image classification system used for generating diagnostic assessments.

15. The apparatus of claim 14, wherein the predefined image classification system includes a standardized system under a class of Reporting and Data Systems (RADS) organized by the American College of Radiology, and the one or more descriptors include-descriptors defined under that standardized system under the class of Reporting and Data Systems (RADS) and by the American College of Radiology.

16. The apparatus of claim 14, wherein the predefined image classification system includes a standardized system under a class of Reporting and Data Systems (RADS) organized by the American College of Radiology, the class including one of: Thyroid Imaging Reporting and Data System (TI-RADS), Breast Imaging Reporting and Data System (BI-RADS), Colonography Reporting and Data System (C-RADS), Liver Imaging Reporting and Data System (BI-RADS), Lung Imaging Reporting and Data System (Lung-RADS), Neck Imaging Reporting and Data System (NI-RADS), Ovarian-Adnexal Imaging Reporting and Data System (O-RADS), and Prostrate Imaging Reporting and Data System (PI-RADS).

17. A method, comprising:
receiving, at a compute device, image data associated with a region of interest;
receiving, at the compute device, a first diagnostic assessment of the region of interest, the first diagnostic assessment being in a first format;
generating feature vectors associated with the image data, the feature vectors configured to be used to generate a diagnostic assessment of the region of interest associated with the image data;
processing the feature vectors using a machine learning (ML) model to generate an output including a second diagnostic assessment of the region of interest, the second diagnostic assessment being in a second format different from the first format;
applying a transformation function to the second diagnostic assessment, the transformation function configured to transform the second diagnostic assessment from the first format to the second format; and
determining, based on the applying the transformation function, a third diagnostic assessment of the region of interest.

18. The method of claim 17, wherein the first diagnostic assessment includes a first set of values associated with one or more descriptors associated with the image data and the third diagnostic assessment includes a second set of values associated with the one or more descriptors associated with the image data, such that the transformation function is configured to transform data included in the second diagnostic assessment in the form of probabilities associated with the image data to generate the second set of values.

19. The method of claim 17, further comprising:
generating a clinical recommendation based on the third diagnostic assessment.

20. The method of claim 19, further comprising:
displaying, responsive to a user request and via an interface, the third diagnostic assessment and the clinical recommendation.
